(19) United States
(12) Patent Application Publication
(10) Pub. No.: US 2019/0382850 A1
(43) Pub. Date: Dec. 19, 2019

(54) METHOD FOR IDENTIFYING MITOCHONDRIAL DNA IN EXTRACELLULAR VESICLES AND TREATMENT OF MTDNA-RELATED DISORDERS AND CANCER

(71) Applicant: Memorial Sloan-Kettering Cancer Center, New York, NY (US)

(72) Inventors: Pasquale SANSONE, New York, NY (US); Jackie BROMBERG, New York, NY (US)

(21) Appl. No.: 16/481,669

(22) PCT Filed: Jan. 26, 2018
(86) PCT No.: PCT/US18/15448

§ 371 Date: Jul. 29, 2019

Related U.S. Application Data
(60) Provisional application No. 62/451,453, filed on Jan. 27, 2017.

(51) Int. Cl. C12Q 1/6886; A61K 35/16

(52) U.S. Cl. CPC C12Q 1/6886; A61K 35/16

(57) ABSTRACT

Isolation of large quantities of mtDNA from cancer patient circulating microvesicles and exosomes as well as cell lines including fibroblasts and bone marrow stromal cells provides a useful biological tool -microvesicles derived mtDNA- to quantify mtDNA, diagnose and study mitochondria disorders and transfer molecules of mtDNA in recipient cells (mtDNA genetic therapy). The treatment of hormonal therapy derived dormant cancer cells with CAF-derived mtDNA^{hi} EVs promotes an escape from metabolic quiescence and hormonal therapy resistant disease both in vitro and in vivo.

This application claims priority from U.S. Provisional Patent Application No. 62/451,453, filed Jan. 27, 2017.

This invention was made with government support under W81XWH-10-1-103, W81XWH-13-0425, W81XWH-13-1-0427 awarded by US Department of Defense; and government support under CA 169538 awarded by National Cancer Institute. The government has certain rights in the invention.

### FIELD

The present application relates generally to mitochondrial DNA in extracellular vesicles as an identifier and functional mediator of phenotypes in cancer and other disorders.

### BACKGROUND

Extracellular vesicles (EVs) are novel mediators of juxtacrine and paracrine signaling required for metastatic progression. Specifically, tumor and stromal cell-derived EVs have been shown to be potent regulators of tumor progression and resistance to therapy by transferring their cargo (proteins, lipids, mRNA, miRNA) into recipient cells promoting signaling cascades and epigenetic changes. Although, cancer cell derived EVs have been shown to regulate pre-metastatic niche formation, organotropism, migration, invasion, sternness and survival little is known about their role in regulating the metabolism of cancers.

Although double stranded DNA has been found in EVs, the presence of the full mtDNA in EVs and the horizontal transfer of this genetic material have never been investigated. MtDNA levels and mutational status within cancers has recently become of interest, as these are associated with the development of a wide-variety of cancers and resistance to therapies. For example, non-tumorigenic cancer cells lacking mtDNA could reacquire tumorigenic potential in vivo following the acquisition of host mtDNA. Complementation of deleterious mtDNA mutations increases tumorigenicity, while expression of such genetic variants or lack of mtDNA impede cancer cell growth.

Though the estrogen receptor (ER) positive variant of breast cancer is touted as the most indolent and favorable, the majority of breast cancer deaths are in fact from this subtype. There are several features of this category of breast cancers that likely account for this outcome. The first is that metastatic relapse can occur many years after initial diagnosis of primary disease. The second is that when metastases do occur, they are invariably in many locations. This observation suggests that these dormant/sleeping metastatic cells are “globally” awakened as if by a “systemic” infection activating the metabolism of these formerly quiescent cells. The third is that once the cancer cells awaken into full-blown metastatic disease, they are largely resistant to ER-directed therapies (i.e. hormonal therapy, HT).

The metabolic features of ER+breast cancer and its evolution from hormonal therapy (HT) sensitive/responsive to dormant and the eventual development of HT-resistant disease is poorly studied in part due to a paucity of pre-clinical models.

### SUMMARY

One aspect of the present application relates to a method for detecting mtDNA molecules in a subject. In one embodiment, the method comprises a reagent for isolating EVs from a biological sample, a reagent for isolating DNA from these isolated EVs, and a reagent for detecting one or more mtDNA genes or the entire genome in isolated DNA.

As a result of the method described above, another aspect of the present application relates to a method for detecting a mtDNA related condition in a subject, comprising the steps of: isolating extracellular vesicles (EVs) from the plasma or other biological fluids, obtained from the subject; detecting the levels of one or more mtDNA genes and the full mtDNA genome in the isolated EVs; identifying specific genetic mtDNA variants; and determining the likelihood of the presence of mtDNA (levels and variants) as a prognostic marker for a number of diseases/conditions including cancer, metabolic disorders, heart failure, brain damage and neurologic syndromes, such as Alzheimer disease.

Another aspect of the present application relates to a method for determining the likelihood of developing resistance to anti-cancer therapies, such as anti-estrogen therapy (endocrine or hormonal therapy, HTR) in breast cancer patients, comprising the steps of: isolating extracellular vesicles (EVs) from the plasma or other biological fluids, obtained from the subject; detecting the levels of one or more mtDNA genes and the full mtDNA genome in the isolated EVs; identifying specific genetic mtDNA variants by DNA sequencing.

Another aspect of the present application relates to a method for detecting a mtDNA related condition in a subject, comprising the steps of: (a) isolating extracellular vesicles (EVs) from the plasma or other biological fluids, obtained from the subject; (b) detecting the levels of one or more mtDNA genes and/or 70%, 80%, 90% or 95% of the full mtDNA genome in the isolated EVs; and determining the likelihood of the presence of a mtDNA related condition based on the result of step (b), wherein the mtDNA related condition is selected from the group consisting of cancer, metabolic disorders, heart failure, brain damage and neurologic syndromes. In another embodiment, the method further comprises the step of (c) identifying specific genetic mtDNA variants; and determining the likelihood of the presence of a mtDNA related condition based on the result of step (b) and step (c), wherein the mtDNA related condition is selected from the group consisting of cancer, metabolic disorders, heart failure, brain damage and neurologic syndromes. In another embodiment , the present application relates to a method for detecting a mtDNA related condition in a subject, comprising the steps of: (a) isolating extracellular vesicles (EVs) from the plasma or other biological fluids, obtained from the subject; (b) identifying specific genetic mtDNA variants; and determining the likelihood of the presence of a mtDNA related condition based on the result of step (b), wherein the mtDNA related condition is selected from the group consisting of cancer, metabolic disorders, heart failure, brain damage and neurologic syndromes.

Another aspect of the present application relates to a method for diagnosing DNA damage in a subject, comprising the steps of: isolating extracellular vesicles (EVs) from the plasma or other biological fluids obtained from the subject; detecting the levels of one or more mtDNA genes and the full mtDNA genome in the isolated EVs; identifying specific genetic mtDNA variants; and determining the likelihood of the presence of mtDNA (levels and variants) as a prognostic marker for a number of diseases functionally associated with oxidative damage including chemotherapy, radiotherapy, neurological syndromes, heart failure, brain damage from chemotherapy, such as chemobrain, and lung/renal damage from doxorubicin and/or radiotherapy administration in cancer and non-cancer patients.

Another aspect of the present application relates to a method for treating, or reducing the likelihood of developing a mtDNA related condition in a subject. In one embodiment, the present application provides a method for reducing the likelihood of developing resistance to hormonal-therapy and escape from tumor dormancy in patients with breast cancer. The method comprises the step of administering to a patient in need of such treatment an effective amount of an agent that inhbits the generation of mtDNA high EVs and the horizontal transfer of mtDNA.

Another aspect of the present application relates to a method for monitoring side effect of a radiation or chemotherapy therapy in a subject, comprising the steps of: (a) isolating extracellular vesicles (EVs) from a biological fluid sample obtained from the subject; (b) detecting the levels of one or more mtDNA genes and the full mtDNA genome in the isolated EVs; and determining the likelihood of the presence of a condition resulted from therapy induced oxidative damge to mtDNA, wherein the condition is selected from the group consisting of neurological syndromes, heart failure, brain damage and lung/renal damage. In one embodiment, the method further comprises the step of (c) identifying specific genetic mtDNA variants in the isolated EVs; and determining the likelihood of the presence of a condition resulted from therapy induced oxidative damge to mtDNA, wherein the condition is selected from the group consisting of neurological syndromes, heart failure, brain damage and lung/renal damage. In a further embodiment, the present application relates to a method for monitoring side effect of a radiation or chemotherapy therapy in a subject, comprising the steps of: (a) isolating extracellular vesicles (EVs) from a biological fluid sample obtained from the subject; (b) identifying specific genetic mtDNA variants in the isolated EVs; and determining the likelihood of the presence of a condition resulted from therapy induced oxidative damge to mtDNA, wherein the condition is selected from the group consisting of neurological syndromes, heart failure, brain damage and lung/renal damage.

Another aspect of the present application relates to a method for determining the likelihood of developing resistance to therapies, such as anti-estrogen therapy (endocrine or hormonal therapy, HTR) in breast cancer patients, comprising the steps of: isolating extracellular vesicles (EVs) from the plasma or other biological fluids, obtained from the subject; detecting the levels of one or more mtDNA genes and the full mtDNA genome in the isolated EVs; identifying specific genetic mtDNA variants. In one embodiment, the present application relates to a method for determining the likelihood of developing hormonal-therapy resistance in a breast cancer patient, comprising the steps of: (a) isolating extracellular vesicles (EVs) from a sample obtained from the subject; (b) detecting the levels of one or more mtDNA genes and the full mtDNA genome in the isolated EVs; and determining the likelihood of developing hormonal-therapy resistance in a breast cancer patient based on the result of the detecting step (b). In a further embodiment, the method further comprises the step of (c) identifying specific genetic mtDNA variants and wherein the likelihood of developing hormonal-therapy resistance in a breast cancer patient is determined based on the result of the detecting step (b) and step (c). In another embodiment, the present application relates to a method for determining the likelihood of developing hormonal-therapy resistance in a breast cancer patient, comprising the steps of: (a) isolating extracellular vesicles (EVs) from a biological fluid sample obtained from the subject; (b) identifying specific genetic mtDNA variants in the isolated EVs; and determining the likelihood of developing hormonal-therapy resistance in a breast cancer patient based on the result of the identifying step (b).

Another aspect of the present application relates to a method for treating, or reducing the likelihood of developing, a mtDNA related condition in a subject. In one embodiment, the present application provides a method for reducing the likelihood of developing resistance to hormonal-therapy and escape from tumor dormancy in patients with breast cancer. The method comprises the step of administering to a patient in need of such treatment an effective amount of an agent that inhbits the generation of mtDNA high EVs and the horizontal transfer of mtDNA.

Another aspect of the present application relates to a method for diagnosing new mtDNA related diseases in a subject: said method comprising the steps of: isolating extracellular vesicles (EVs) from the plasma or other biological fluids, obtained from the subject suffering from distinct diseases which have not yet been related to mtDNA pathogenesis (metastatic cancer, metabolic sydromes, autoimmune disorders, inflammatory syndromes, neurodegenerative diseases); detecting the levels of one or more mtDNA genes and the full mtDNA genome in the isolated EVs; identifying specific genetic mtDNA variants (haplotypes); and determining the likelihood of the presence of mtDNA (levels=copy number and variants=haplotypes) as a prognostic marker(s) for these disorders. In certain embodiments, the disease is selected from the group consisting of an autoimmune disease, an inflammatory disease and a neurodegenerative disease. In further embodiments, the autoimmune disease is selected from the group consisting of Churg-Strauss Syndrome, Coeliac disease, Hashimoto's thyroiditis, Goodpasture Syndrome, Graves' disease, inflammatory bowel disease, psoriasis, psoriatic arthritis, rheumatoid arthritis (RA), Sjogren's syndrome and systemic lupus erythematosus (SLE); wherein said inflammatory disease is selected from the group consisting of allergy, amyotrophic lateral sclerosis (ALS), asthma, chronic inflammatory disorder, atopic dermatitis, coronary atherosclerosis, interstitial cystitis, diabetes mellitus type 1 (IDDM), idiopathic thrombocytopenic purpura, multiple sclerosis and chronic pancreatitis; and wherein said neurodegenerative disease is selected from the group consisting of autism spectrum disorders (ASD), chronic fatigue syndrome, chronic prostatitis, fibromyalgia, vitiligo and Parkinson's Disease. In other embodiments, the disease is selected from the group consisting of rheumatoid arthritis, psoriasis, autism spectrum disorders (ASD), SLE and mastocytosis and cancers. In some embodiment, metastatic cancer disease is selected from the group of solid tumors spreading to visceral organs including liver, lungs, bones and lymph nodes (breast, head and neck, ovarian, lung, colon, gastric, melanoma and etc.).

Another aspect of the present application relates to the possibility of complementing or rescuing a mtDNA related disorder in humans by the administration of EVs loaded with wild type or functional mtDNA molecules (any genetic variant of mtDNA which promotes higher mitochondrial activity). In certain embodiments, this is a method to rescue mtDNA deficiency related disease (decreased mtDNA copy number) or complement aberrant mtDNA genetic variant related disease in humans: mtDNA genetic therapy, comprising the steps of: diagnosing a mtDNA related disorder in a subject (mtDNA level and genetic variants in the subject's EVs and tissue); isolating EVs from the subject; generating EVs loaded with mtDNA (wild type or a specific variant which is required to rescue or complement mitochondrial activity) by genetic insertion of recombinant mtDNA in subject's EVs; and administration of these functional mtDNA high EVs in the subject either in the circulation (plasma) and/or topically (intra-tissual).

Another aspect of the present application relates to a kit for detecting a mtDNA related condition in a subject from cancer, metabolic disorders to neuropathies. In one embodiment, the kit comprises a reagent for isolating EVs from a biological sample, a reagent for isolating DNA from isolated EVs, and a reagent for detecting one or more mtDNA genes or the entire genome in isolated DNA. In some embodiments, the kit further comprises a filter for isolating EVs from the biological sample.

### BRIEF DESCRIPTION OF THE DRAWINGS

The present invention can be better understood by reference to the following drawings, wherein like references numerals represent like elements. The drawings are merely exemplary to illustrate certain features that may be used singularly or in any combination with other features and the present invention should not be limited to the embodiments shown.

FIG. 1 shows mitochondrial genome identified in EVs from the plasma of patients with HTR disease. (Panel a) Circulating EVs were isolated from the plasma (5-10 ml) of 1) 22 patients with HTR metastatic disease. Patients with high volume disease (>10% of organ involvement) were denoted in red and low volume disease (<1% of organ involvement) denoted in blue 2) 9 healthy controls; 3) 12 patients with early stage breast cancer following removal of their cancer; 4) 6 patients with de novo metastatic breast cancer who had not yet received treatment. DNA was isolated and mtDNA copy number quantification was determined by qPCR for the ND1 gene (2 ng of DNA was used). Representative NanoSight plot (mode and size) and electron micrographs are also shown (Patient.6). Data are reported as mean±s.d performed in triplicate; each point represents a patient value. *P<0.005 (Student's t-test). (Panel b) Schematic and representative gel electrophoresis image of long range PCR (3 contiguous amplicons: Mito1-3.9 kb, Mito2-5.5 kb, Mito3-7.8 kb) encoding the complete 16.6 kb circular mitochondrial genome purified from EVs in the plasma of Pt. 29 (2 ng of total DNA). (Panel c) Schematic and representative gel electrophoresis image of whole genome amplification (using 46 overlapping PCR amplicons covering the complete mtDNA genome) from patient-derived EV-DNA (ing for each PCR, EV-Patient. 24).

FIG. 2 shows the horizontal transfer of host (murine) mtDNA associates with HTR disease. (Panel a) Representative tumor growth kinetics (BLI) of GFP+/Luciferase+MCF7 xenografts (n=10) treated with weekly fulvestrant once mammary fat pad tumors were established (after 3 months) which led to a period (4 months) of disease stability on HT (HTS) followed by exponential growth on HT (HTR). Representative H&E images of tumors and primary cultures from unsorted as well as FACS purified cancer cells (GFP+/Epcam+) and murine CAFs (GFP−/Epcam). Scale bar 20 μm. (Panel b) OXPHOS potential in tumor-derived cancer cells (panel a) was measured±HT (fulvestrant, 10 μM) by Seahorse. (Panel c) mtDNA/nDNA (human and murine) level as Fold change (log₁₀scale) in cancer cells and mCAFs isolated from HTS and HTR disease (panel a). (Panel d) Percentage of metastatic lesions isolated by FACS from tumor-bearing mice (n=8/group) expressing murine mtDNA as determined by ND1 qPCR (2 ng of total DNA). Oxygen consumption rates (OCR) in murine-mtDNApos and ctrl HTR cells determined by Seahorse. (Panel e) Murine mitochondrial RNA expression (12 genes: ND1, ND2, COX2, ATP8, ATP6, COX3, ND3, ND4L, ND4, ND5, ND6, CytB) as fold change (logᵢₒscale) determined by qRT-PCR in HTR and HTS cells (panel a). (Panel f) Proliferation potential in presence of mitochondrial poisons in HTR cells (atovaquone 1 μM, rotenone 100 nM, ρ0 media, oligomycin 200 nM) and ±HT. Error bars, mean±s.d. of BLI from n=3 samples. Data are reported as error bars, mean±s.d. of n=3 independent experiments (b-f); *P<0.05 (Student's t-test).

FIG. 3 shows stromal-derived Extracellular vesicles (EVs) harbor the mitochondrial genome. (Panel a) qPCR of Murine (Mu)-mtDNA ND1 in circulating EV DNA isolated from HTR and HTS tumor-bearing mice (n=3/group). (Panel b) Electron microscopy (scale bar, 500 nm) and NanoSight analyses of extracellular-vesicles (EVs) isolated from mCAFs. (Panel c) Representative exosomal proteins identified by quantitative mass spectrometry of mCAF derived EVs. (Panel d) Ratio of mtDNA/nDNA level by qPCR (2 unique primer sets for Murine-ND1; one for Human-ND1, Murine-GAPDH and Human-GAPDH) in EVs (isolated from mCAFs and human bone marrow stromal cells -H-BMSCs-, HS27a)±DNAse0 treatment (to eliminate exogenous DNA contamination, see methods); error bars, mean±s.d. of n=3 independent experiments; *P<0.05 (Student's t-test). Representative electron microscopy image of HS27a EVs; scale bar 500 nm. (Panel e) Schematic and representative PCR gel electrophoresis of mtDNA (ND1) and nDNA (GAPDH) in floating fractions/pellet from a sucrose gradient (see methods) of mCAF derived EVs. Free DNA was eliminated by DNAse0 digestion previous extraction of EV-DNA. Western blot analysis of EV s -CD63- and mitochondria -ATP5A1- are also reported. (Panel f) mtDNA level as absolute copy number (qPCR, ND1) from HS27a EVs and cells. (Panel g) Schematic and representative gel electrophoresis image of long range PCR (3 contiguous amplicons) encoding the 16 kbp mtDNA genome from purified EVs±DNase0. (Panel h) Electropherogram of the DNA sequence of ND1 gene showing a mutation conserved between HS27a cells and EVs.

FIG. 4 shows mCAF-derived EVs educate tumor cells mediating HTR disease. (Panel a) Murine mtDNA copy number by qPCR in mCAFs and their EVs; Wt, wild type; ρ0, cells depleted for mtDNA (see methods). Bar graph reports mean±s.d of copy number (log₁₀scale) of n=3 independent experiments. (Panel b) Schematic: HT-sensitive (HTS-green) cells were treated with 3×10⁹ mCAF-derived EVs (wt-mtDNA^{hi} or ρ0-mtDNA^{lo}) weekly ×4+HT (fulvestrant, 10 μM/weekly) leading to the growth with wt-mtDNA^{hi} EV educated cells. (Panel c) Proliferation (by CalceinAM) of BT474 (HTS cells) cultured for 24 days±HT (fulvestrant, 10 μM/weekly) treated weekly with 3×10⁹ mCAF-derived EVs (wt-mtDNA^{hi} or ρ0-mtDNA^{lo}). The mean±s.d is reported for each time point of the growth curve; *P<0.05 (post-hoc t-test corrected for multiple comparisons after GLM for repeated measures). (Panel d) Schematic and tumor growth curve of HTS cells (MCF7) injected in the MFP and subsequently educated weekly (retro-orbital injection-3×10⁹) with either wt-mito^{hi} or ρ0-mtDNA^{lo} EVs. After 8 weeks, HT was administered (fulvestrant-100 μg/mouse/weekly) for 6 weeks. The mean±s.e.m is reported for each time point of the growth curve; *P<0.05 (post-hoc t-test corrected for multiple comparisons after GLM for repeated measures).

FIG. 5 shows mCAF-derived EVs promote the exit from HT-induced tumor dormancy. (Panel a) Schematic of the experimental design and representative flow plots: HT naive cells were FACS isolated from xenografts (GFP+) or luminal breast cancer cell lines, treated with HT (fulvestrant, 10 μM/weekly for 2 months); HT dormant cancer cells (6% of viable population, HTDorm) were FACS purified (Dapi-) and displayed a single cell morphology in 3D (non proliferating, scale bar 100 μm). (Panel b) mtDNA level (qPCR as fold change, naïve cells was used as reference) in luminal breast cancer cells and xenograft derived cells (multiple models)±HT (fulvestrant, 10 μM for 2 months). (Panel c) Dormant MCF7 cells (HTD) were isolated and treated with 3×10⁹ mCAF-derived EVs (wt-mtDNA^{hi} or ρ0-mtDNA^{lo}) weekly ×4+HT (fulvestrant, 10 μM/weekly). After 40 days, mammosphere (MS) number and mitochondrial membrane potential (Δψ) by TMRE staining (red) in HTR/mito^{hi} EV and HTD/mito^{lo} EV educated cells was determined; scale bar 15 μm. (Panel d) Confocal microscopy of HTD cells incubated with labeled mCAF EVs (PKH67, green for 48 h), mitochondria (Mitotraker, red) and co-localization of EVs with mitochondria (yellow); scale bar 5 μm. (Panel e) Murine mtDNA level (qPCR ND1, as fold increase log₁₀scale of reference HTD) from two HTD-HTR models described in panel d (MCF7 and BT474). (Panel f) Murine mitochondrial RNA expression (14 genes, as fold change -log₁₀scale-) by qRT-PCR in HTR and HTS cells (panel e) (reference HTDorm cells; MCF7); the expression of nuclear encoded murine (non-mitochondrial RNA) transcripts was also determined (Cox4, β2M). (Panel g) Schematic and representative BLI and bar graph of tumor growth derived from HTD cells (ZR751 GFP+/Luciferase+cells) injected in the MFP of mice (n=5/group) and treated (weekly retro-orbital injection ×8) with 3×10⁹ wt-mito^{hi} or ρ0-mito^{lo} mCAF derived EVs; error bars, mean±s.d. after 4 months; *P<0.05 (Student's t-test). Mu-mtDNA level is reported as copy number (qPCR ND1) in FACS purified ZR751-EV educated cells at the endpoint of the experiment. (Panel h) Representative gel electrophoresis from whole murine mtDNA PCR amplification using a set of NumtS (nuclear mitochondrial sequences)-excluding overlapping primers in cancer cells from panel g (see methods). Data are reported as error bars, mean±s.d. of n=3 independent experiments (b, c, e-g); *P<0.05 (Student's t-test).

FIG. 6 shows the schematic of the patient and experimental model described in this patent: EV-mediated horizontal transfer of mtDNA promotes HTR disease and exit from therapy-induced dormancy. The horizontal transfer of mtDNA promotes hormonal therapy resistance. Extracellular vesicles (EVs) from cancer-associated fibroblasts (CAFs) contain the whole mtDNA genome, which is transferred to HT-derived dormant or HT-sensitive cancer cells promoting mitochondrial activity (OXPHOS) and HTR disease.

FIG. 7 shows that circulating EV DNA from therapy resistant patients is enriched for whole mtDNA genome. (Panel a), Dot plot of nuclear DNA copy number quantification as determined by qPCR for GAPDH gene in circulating EVs isolated from fresh collected plasma (5-8 ml) of 8 healthy controls and 20 luminal breast cancer patients at different stage of their disease (Table 5). Each point corresponds to a patient sample. (Panel b), Schematic and gel electrophoresis of whole mtDNA genome amplified in several patient derived circulating EV-DNA resulting from 46 overlapping PCR amplicons to cover all the mtDNA genome. (Panel c), mtDNA ND1 level change (Ct qPCR value from 2 ng of total EV-DNA) determined in the same patient (two different examples are reported) following time and therapy response (PR, partial response; Stable Disease; POD, progression of disease); (Panel d) mtDNA ND1 level change (Ct qPCR value from 2 ng of total EV-DNA) determined in two different patients (#1, #2) before and after a certain therapy. Data are reported as error bars, mean±s.d. of n=3 independent experiments.

FIG. 8 reports that OXPHOS inhibition abrogates HTR disease. (Panel a), Proliferation potential as determined by in vitro cell growth (CalceinAM) in luminal (ER+) and triple negative cancer cell lines cultured in media with increasing concentration of glucose. (Panel b), Oxygen consumption rates (OCR)±oligomycin (200 nM)/rotenone (100 nM) in MCF7 and MDA-MB-231 cells as determined by seahorse technology. (Panel c), Desmin immunohistochemical images of CAFs in HTS/HTR-derived tissues (scale bar 25 μm). (Panel d), Schematic analysis of the generation of metastatic HTR disease in mice. Following 4 months from primary tumor xenografts (MCF7), tumor tissues were removed and mice were randomized to receive HT (fulvestrant, 100 mg/injection/weekly). Metastatic burden was analyzed by BLI and at necropsy by histological examination (H&E). (Panel e), Proliferation potential in presence of oligomycin (100 nM) and ±HT as determined by in vitro BLI in metastatic cells from HTS and HTR cells FACS purified from xenografts (FIG. 2a). Error bars, mean±s.d. of BLI from n=3 samples. (Panel f), mtDNA/nDNA level in circulating EVs from HTR xenografts as determined by copy number (GAPDH, ND1: qPCR Fold change). Data are reported as error bars, mean±s.d. of n=3 independent experiments (a, e-g). *P<0.05 (Student's t-test) and GLM anova after multiple comparisons (a).

FIG. 9 reports that the entire mtDNA genome is present in EVs. (Panel a), Bar graph showing human mtDNA copy number in MCF7 cells and EVs from two different experiments. (Panel b), Bar graph of human mtDNA copy number in EV isolated from the conditioned media of human cancer cells and fibroblasts (5×10⁷ cells). CL1-3, xenograft derived MCF7 derivatives; HMF, normal mammary gland fibroblasts; MRC5, normal lung fibroblasts; CAFs SP, patient derived cancer associated fibroblast cultures. (Panel c), Representative gel electrophoresis images of mtDNA PCR amplicons Mito1/2 derived from EV-DNA. Pos CTRL, cellular derived DNA (HS27a). (Panel d), Gel electrophoresis of whole mtDNA genome amplified in EV-DNA resulting from 46 overlapping PCR amplicons to cover all the mtDNA genome from multiple fibroblast derived cultures and cancer cell lines. (Panel e), Table representing mtDNA mutation and SNPs in EVs and cells from HS27a and HMF cell lines. Data are reported as error bars, mean±s.d. of n=3 independent experiments (a); *P<0.05 (Student's t-test).

FIG. 10 shows that depletion of mitochondria DNA in CAFs hampered the EV-dependent up-regulation of OXPHOS potential in HT cells. (Panel a), Proliferation potential as determined by in vitro cell growth (CalceinAM) in mCAFs (wild type or ρ0). (Panel b), Mitochondrial transcriptome as determined by qPCR in mCAFs cells (wild type or ρ0). Error bars, mean±s.d of fold change (reference wild type). *P<0.05 (Student's t-test). (Panel c), OXPHOS potential in cancer cells±HT (fulvestrant, 10 mM) administered for 48 hours with mite-EVs and mito^{hi} EVs (10¹³EVs for 10⁶ cells). Data are reported as error bars, mean±s.d of respiration rate under mitochondrial stressors (FCCP, oligomycin, rotenone). *P<0.05 (Student's t-test). (Panel d), mtDNA level (qPCR, ND1) in HTS and HTR cells±HT (fulvestrant, 10 mM). Error bars, mean±s.d.of fold change (reference HTS). *P<0.05 (Student's t-test).

FIG. 11 shows HT induced metabolic dormancy and reduction of mtDNA level. (Panel a), Representative bright field images of MCF7 po cells±CAF-EVs (3×10⁹/weekly for 40 days). Bar graph of number of MS is also reported at the endpoint of the experiment (40 days). (Panel b), HT led to decreased expression of mRNA involved in protein synthesis and OXPHOS signaling with z score and -log (p-values)- as determined by Ingenuity analysis of microarray data from RNA isolated FIG. 5a (MCF7), single P values are also reported. (Panel c), OXPHOS potential determined as changes of respiration rate under mitochondrial stressors by seahorse technology (oligomycin 1 μM, rotenone 100 nM) in cancer cells isolated from FIG. 5b (MCF7, PT1). (Panel d), Western Blot analysis of OXPHOS protein cocktail in cells from panel c. (Panel e), Mitochondrial Complex I/IV activity quantification as determined by nmol/min/mg of protein in luminal breast cancer cell lines from panel a. Data are reported as error bars, mean±s.d. of n=3 independent experiments (c, e); *P<0.05 (Student's t-test).

FIG. 12 Mito^{hi} EV educated HTR cells display proficient mitochondria. (Panel a), Confocal Microscopy images of HTD cells (MCF7) following labeled CAF-EVs administration: CAF-EVs were isolated from 3×10⁹ mCAFs, labeled with PHK26 green (according to manufacturer's protocol) and EtBr (1.5 ng); PHK26^{pos}/EtBr^{pos} EVs were than administered to cancer cells and 24 h later confocal imaging was performed. Yellow arrows show co-localization of DNA and EVs. Scale bar 5 mM. (Panel b), qPCR analysis of murine mtDNA level (ND1 as fold change of ND1 level from mCAF) from MCF7 cells cultured in vitro and in vivo administered with mCAF-EVs (3×10⁹/weekly/month). (Panel c), Mitochondria morphology as determined by electron microscopy in the MCF7 model: cancer cells in absence of HT (control) and following HT-induced dormancy (HTD)±CAF-EVs (mito^{lo} or mito^{hi}). Arrows shows damaged (loss of cristae, matrix-dense round mitochondria) and healthy mitochondria (control). Scale bar 200 nm. (Panel d), Mitochondrial Complex I/IV activity quantification as determined by nmol/min/mg of protein in luminal breast cancer cell lines from panel a. Data are reported as error bars, mean±s.d. of n=3 independent experiments, *P<0.05 (Student's t-test).

FIG. 13 shows that depletion of mtDNA abrogates the CAF-dependent tumorigenic potential of HTS cells. (Panel a), Representative electropherograms of murine ND1 and ND5 sequences (amplicons 36, 16, 11, 10) derived from whole murine mtDNA sequencing using a set of NumtS (nuclear mitochondrial sequences)-excluding overlapping primers in cancer cells from FIG. 5h (see methods). (Panel b), Dot plots of murine mtDNA level (ND1 copy number) from FACS purified cancer cells derived from xenografts tissues of EV-treated mice and cultured in vitro for one month (2 different examples xeno#1 and xeno#2 and control are reported). Data are reported as error bars, mean±s.d. of n=3 samples. (Panel c), Schematic of the experimental design: HTS cells (MCF7, Luciferase^{pos}) were originated in vitro following chronic fulvestrant administration (fulvestrant, 10 μM/weekly for 2 months), were injected (10⁵) into the MFP of mice in presence/absence of mCAFs (wild type or p0: 1:10=CAFs:MCF7). Tumor growth as BLI mean±s.d. was determined at the endpoint of the experiment (5 months from the injection, n=10 mice/group). *P<0.05 (Student's t-test). (Panel d), Murine mtDNA expression as mean±s.d. copy number (ND1, qPCR) in xenograft derived cancer cells FACS (GFP+) isolated at the endpoint of experiment panel c. Representative gel electrophoresis images of murine mtDNA sequences (ND1, COX3, ND5) expressed in tumor cells (distinct lesions) purified via FACS from xenografts (panel c). Data are reported as error bars, mean±s.d. of n=3 samples. *P<0.05 (Student's t-test).

FIG. 14 reports that hypoxia reoxygenation culture condition increases EV mtDNA copy number in a Jak2-Stat(s) dependent manner. 10-cm plates with 10⁶ cells (CAFs) were cultured in 1% O₂ (hypoxia) for 4 days and 3 days in normal culture conditions (20% O₂, hyperoxia) in presence/absence of Jak2 inhibitor (500 nM every 3 days). Conditioned media for EV isolation and DNA level from EVs was analyzed. As control, EVs from 20% O₂, hyperoxia were also collected and analyzed. MtDNA as copy number is shown as bar graphs, mean±error bars of ND1 level obtained from EV-DNA (10 ng total DNA).

### DETAILED DESCRIPTION

The following detailed description is presented to enable any person skilled in the art to make and use the object of this application. For purposes of explanation, specific nomenclature is set forth to provide a thorough understanding of the present application. However, it will be apparent to one skilled in the art that these specific details are not required to practice the subject of this application. Descriptions of specific applications are provided only as representative examples. The present application is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. Headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). The terms “a” and “an” herein do not denote a limitation of quantity, but rather denote the presence of at least one of the referenced items.

As used herein, the term “mtDNA-related condition” refers to a condition that is caused by, or related to, (1) mutation or deletion in mtDNA, (2) horizontal mtDNA transfer and (3) destruction and/or loss of mtDNA. Examples of conditions that are caused by, or related to, mutation or deletion in mtDNA include but are not limited to, cancer, Kearns-Sayre syndrome (KSS), Leber Hereditary Optic Neuropathy (LHON), subacute sclerosing encephalopathy, progressive external ophthalmoplegia, Pearson Syndrome, Leigh syndrome, exercise-induced muscle pain, fatigue and rhabdomyolysis, amino-glycoside-induced hearing loss, mitochondrial myopathy, encephalopathy, lactic acidosis and stroke-like episodes (MELAS), biventricular cardiac hypertrophy, diabetes mellitus and deafness, strokes and migraine and seizures and ataxia, retinitis pigmetosa, and ptosis (NARP), lactic acidaemia in children, myoclonic epilepsy with ragged red fibers that is associated with ragged-red fibres, Alzheimer disease, Parkinson disease, myoneurogenic gastrointestinal encephalopathy (MNGIE) and others (see, e.g., Nat Rev Genet. 2005 May; 6(5):389-402; Walker and Chalkia, Cold Spring Harb Perspect Biol. 2013 Nov 1;5(11): a021220. doi: 10.1101/cshperspect.a021220). Examples of conditions that are caused by, or related to, horizontal mtDNA transfer include but are not limited to, acquirement of resistance to hormonal therapy in breast cancer cells. Examples of conditions that are caused by, or related to, destruction and/or loss of mtDNA include but are not limited to, cancer, cardiac failure, brain damage such as chemo brain, and other conditions caused by chemo therapy or radiation therapy.

As used herein, the term “full mtDNA genome” refers to the total mtDNA sequences in a sample. In some embodiments, the term “full mtDNA genome” refers to 70%, 80%, 90% or 95% of the total genomic mtDNA sequence in a sample.

As used herein, the term “specific genetic mtDNA variants” refers to haplotypes of mtDNA.

The model herein differs from xenocybrids for several reasons: 1) it does not use enucleated donor cells which function as “mitochondrial” donors containing full mitochondria, cytoplasm and organelles which fuse with recipient cells; 2) the recipient cells are not mtDNA depleted but have reduced mtDNA from hormonal therapy; 3) experiments are performed with EV which are vehicles capable of transferring genomic DNA from cell type to the next conferring profound phenotypes including transformation; 4) EVs fuse with resident mitochondria and are known to traffic to mitochondria via Rabs. This experimental model demonstrates the transfer of exogenous mtDNA in vivo via EVs and provides its functional consequence in OXPHOS dependent breast cancers.

Methods for Detecting a mtDNA Related Condition

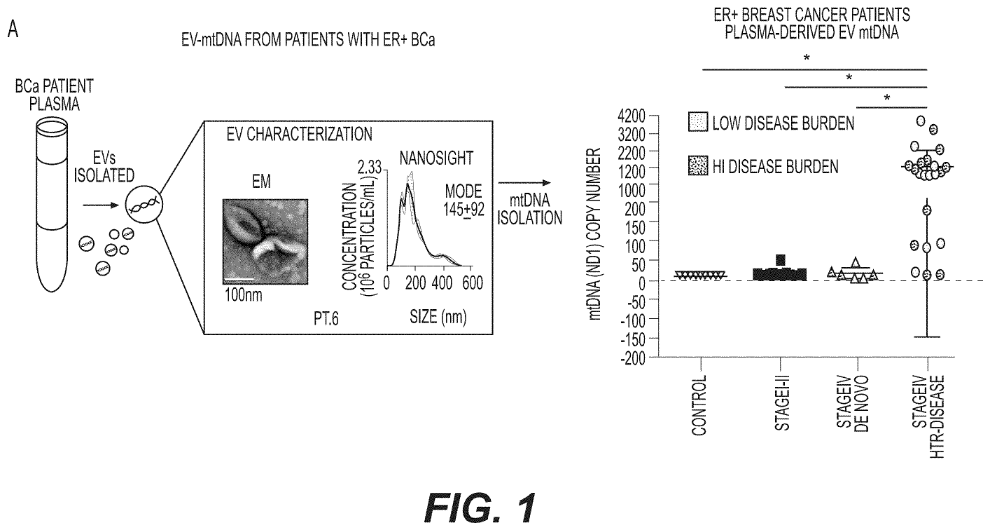

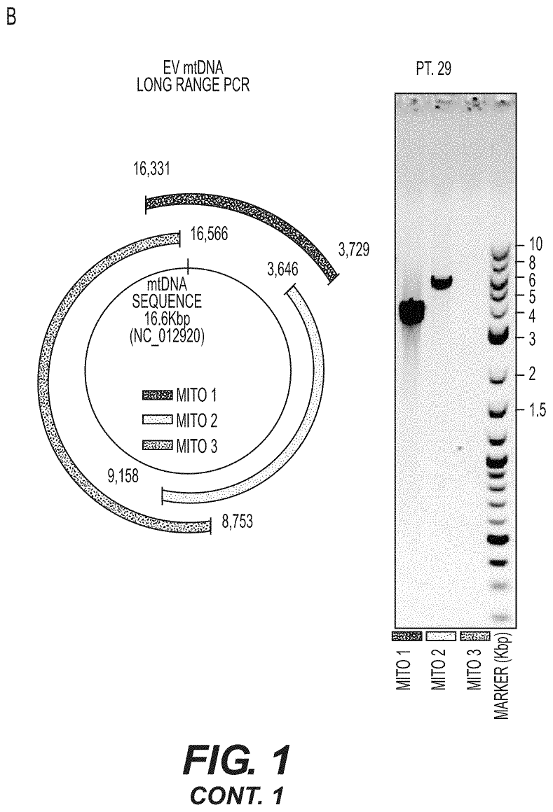

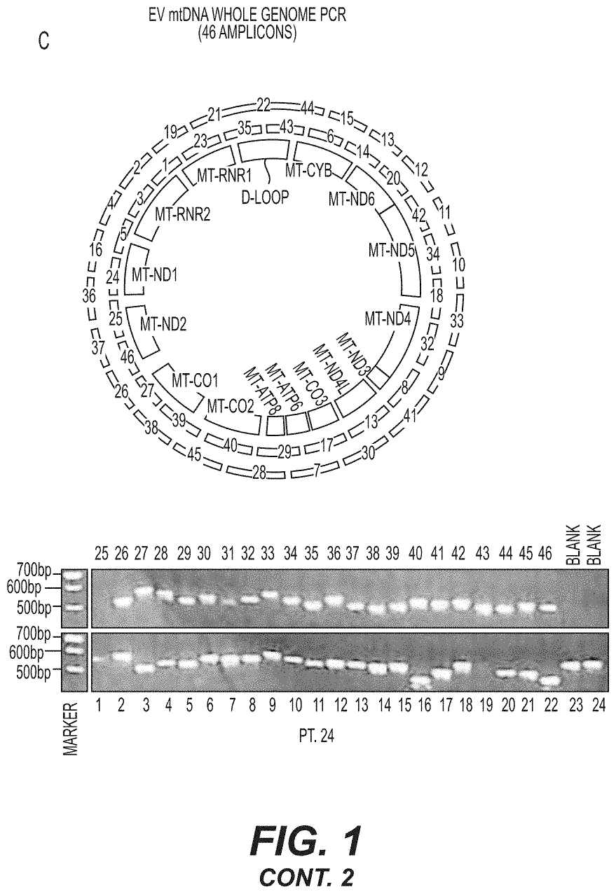

One aspect of the present application relates to a method for detecting a mtDNA related condition in a subject. The method includes the steps of isolating extracellular vesicles (EVs) from the plasma of a subject, detecting not just one gene but the presence of 70%, 80%, 90% or 95% or the entire mtDNA genome (using two methods: long range per and whole mtDNA nested PCR) and determining the level (copy number by semi quantitative PCR) and likelihood of the presence of genetic alterations in the mtDNA (by SNP analysis) in the subject based on the result of the detection step (FIGS. 1-3 and 7-9: isolation of mtDNA from EVs of breast cancer patients and experimental models of breast cancer). In one embodiment, the method includes the steps of 1. isolating extracellular vesicles (EVs) from the plasma or other biological fluids, obtained from the subject; 2. detecting the levels of one or more mtDNA genes and the full mtDNA genome in the isolated EVs; 3.identifying specific genetic mtDNA variants; 4. and determining the likelihood of the presence of mtDNA (levels and variants) as a prognostic marker for a number of diseases including metabolic disorders, heart failure, neurologic syndromes (e.g. Alzheimer) and cancer. In one embodiment, the method may rely on detecting levels. In another embodiment, the method may rely on identifying variants.

Examples of conditions that are caused by, or related to, horizontal mtDNA transfer, include but are not limited to, cancer, development of resistance to hormone-therapy in breast cancer and metabolic disorders (see, e.g., Nat Rev Genet. 2005 May; 6(5):389-402; Walker and Chalkia, Cold Spring Harb Perspect Biol. 2013 Nov. 1;5(11): a021220. doi: 10.1101/cshperspect.a021220). As used herein after, the term “horizontal mtDNA transfer” refers to transfer of mtDNA between two cells via dynamic intercellular organelle highways (e.g., via extracellular vesicles) or nanotubes.

The EVs include, but are not limited to, microvesicles, ectosomes, shedding vesicles (shed microvesicles (sMVs)), microparticles, and exosomes. EVs are often isolated from a biosample, such as plasma or urine by differential centrifugation, filtration or combinations thereof. Additional purification can be achieved by immunoadsorption using a protein of interest, which also selects for vesicles with an exoplasmic or outward orientation. Isolation strategies typically used, include differential centrifugation (DC), density-gradient centrifugation (DGC), sucrose cushion centrifugation, HPLC gel-permeation chromatography (GPC), affinity capture (AC), microfluidic devices (e.g., trapping exosomes with an immune-affinity approach, such as Exochip; sieving with nanoporous membranes; trapping exosomes on porous structures, such as nanowire on micropillars), synthetic polymer-based precipitation, and membrane filtration (e.g., stirred ultrafiltration cells; ultrafiltration spin columns/tubes operated using low centrifugal force; nanomembrane ultrafiltration spin devices equipped with low protein-binding membranes).

The sample can be any biosample obtained from the subject, including but not limited to, blood, plasma, serum, urine, lymph, cerebrospinal fluid, colonic fluid, nasal fluid, vaginal secretion, skin biopsy and other tissue biopsy.

In one embodiment, the method comprises the steps of isolating extracellular vesicles (EVs) from a plasma sample obtained from the subject by centrifugation and/or filtration, isolating DNA from isolated EVs, determining the level of mtDNA ND1 gene in the isolated DNA, wherein a presence of mtDNA ND1 gene in an amount that gives a qPCR Ct number in the range of 32-28 for 2 ng of DNA is indicative of the presence of the entire mtDNA, or a tendency of developing therapy resistant metastatic breast cancer and mtDNA related diseases in the subject.

Method for Treating a Condition Relating to Horizontal mtDNA Transfer

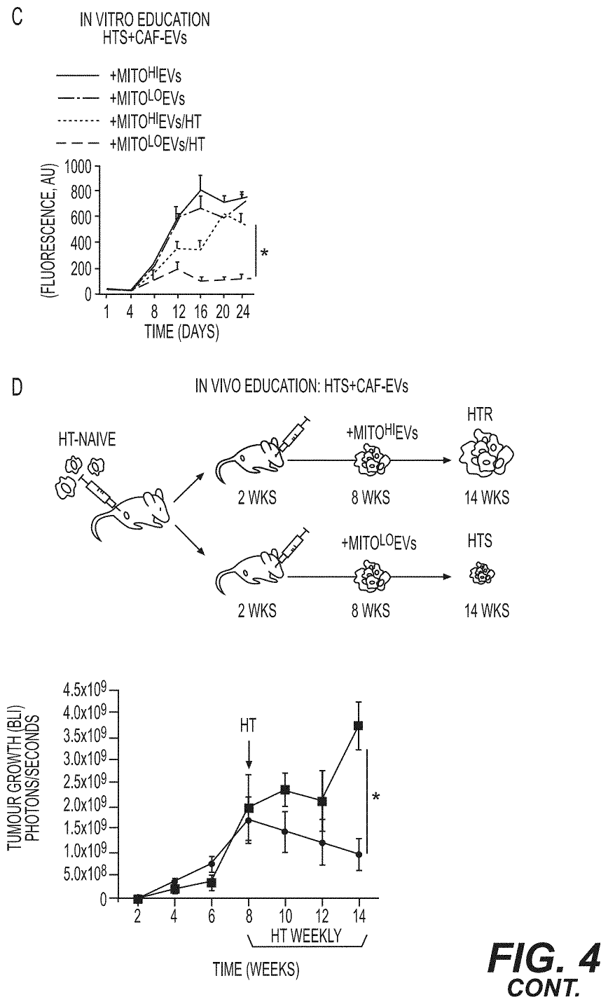

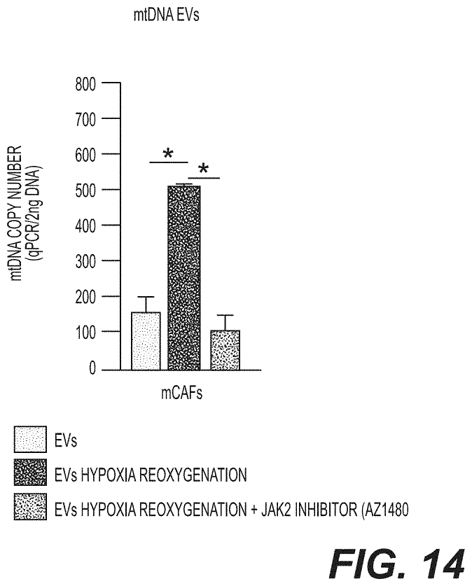

Another aspect of the present application relates to a method for treating or reducing the likelihood of, a medical condition relating to horizontal mtDNA transfer in a subject. The method includes the steps of adminstering to the subject an effective amount of an agent that inhibits horizontal transfer of mtDNA. The method includes the steps of adminstering to the subject an effective amount of an agent that inhbits mtDNA biogenesis such as ethidium bromide, or Jak2 inhibitor (AZD 1490) hampering the generation of mtDNA high EVs and the consequent horizontal transfer of mtDNA from EVs to recipient cells/organs (FIGS. 4-5 and FIGS. 10-13: the functional relevance of mtDNA horizontal transfer in breast cancer, FIG. 14: the role of hypoxia and Jak2 in the biogenesis of mtDNA high EVs).

Examples of agents that inhibit horizontal transfer of mtDNA include, but are not limited to selective blockers of tunneling nanotube formation, such as cytochalasin B, metformin, everolimus, cytarabine, daunorubicin, latrunculin-A, inhibitors of connexin oligomerization (such as inhibitors of connexin-43), as well as inhibitors of RISP, inhibitors of Miro1, hsp90/70 inhibitors, proton pump inhibitors, heparin and inhibitors of NF-KB.

Suitable dosages of the molecules used will depend on the age and weight of the subject and the concentration and/or formulation of the therapeutic composition. As a general proposition, a therapeutically effective amount of the inhibitor of horizontal transfer of mtDNA will be administered in a range from about 1 ng/kg body weight/day to about 100 mg/kg body weight/day whether by one or more administrations. In a particular embodiment, each trispecific inhibitor is administered in the range of from about 1 ng/kg body weight/day to about 10 mg/kg body weight/day, about 1 ng/kg body weight/day to about 1 mg/kg body weight/day, about 1 ng/kg body weight/day to about 100 μg/kg body weight/day, about 1 ng/kg body weight/day to about 10 μg/kg body weight/day, about 1 ng/kg body weight/day to about 1 μg/kg body weight/day, about 1 ng/kg body weight/day to about 100 ng/kg body weight/day, about 1 ng/kg body weight/day to about 10 ng/kg body weight/day, about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg /kg body weight/day to about 1 mg/kg body weight/day, about 1 μg /kg body weight/day to about 100 μg/kg body weight/day, about 1 μg /kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In other embodiments, the inhibitor of horizontal transfer of mtDNA is administered in the range of about 10 ng to about 100 ng per individual administration, about 10 ng to about 1 μg per individual administration, about 10 ng to about 10 μg per individual administration, about 10 ng to about 100 μg per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 μg per individual administration, about 100 ng to about 10 μg per individual administration, about 100 ng to about 100 μg per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per injection, about 1 μg to about 10,000 mg per individual administration, about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per injection, about 10 μg to about 10,000 mg per individual administration, about 100 μg to about 1 mg per individual administration, about 100 μg to about 10 mg per individual administration, about 100 μg to about 100 mg per individual administration, about 100 ng to about 1000 mg per injection, about 100 μg to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The inhibitor of horizontal transfer of mtDNA may be administered daily, every 2, 3, 4, 5, 6 or 7 days, or every 1, 2, 3 or 4 weeks.

In other particular embodiments, the amount of the inhibitor of horizontal transfer of mtDNA may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, age and condition of the patient. Dosages can be tested in several art-accepted animal models suitable for any particular mitochondria-related disorder.

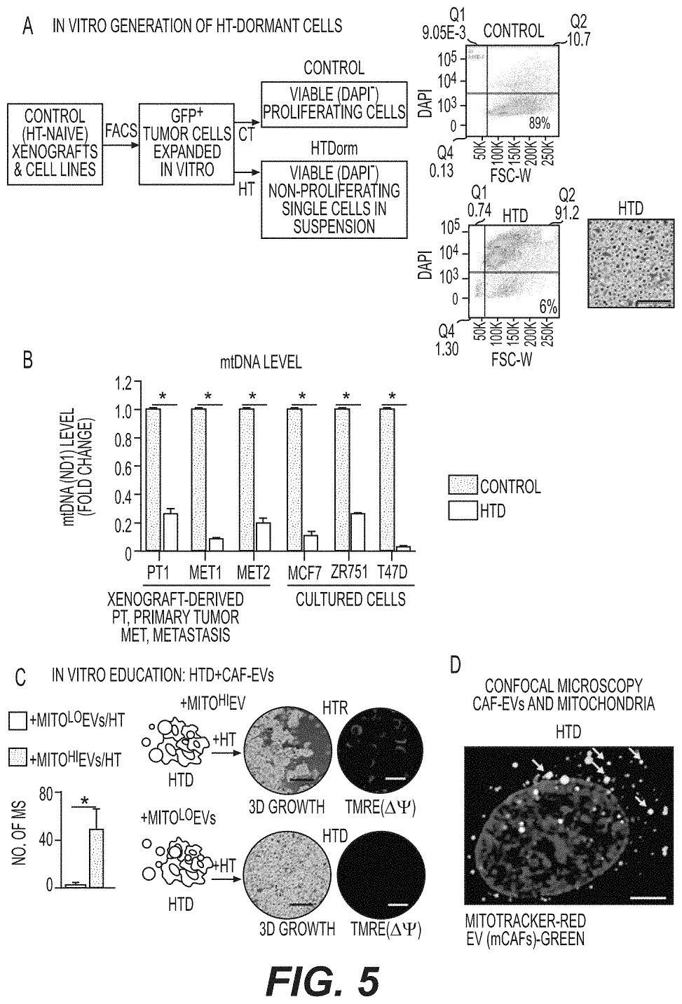

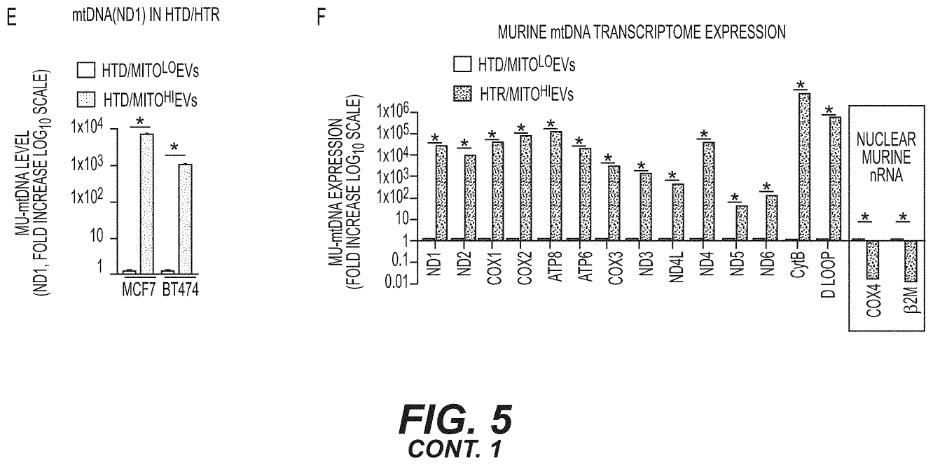

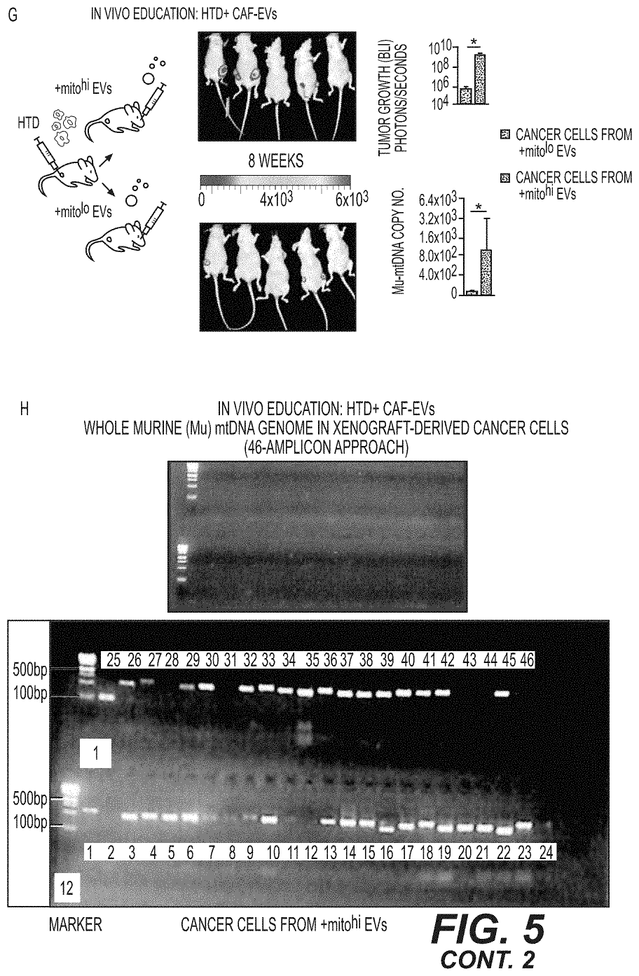

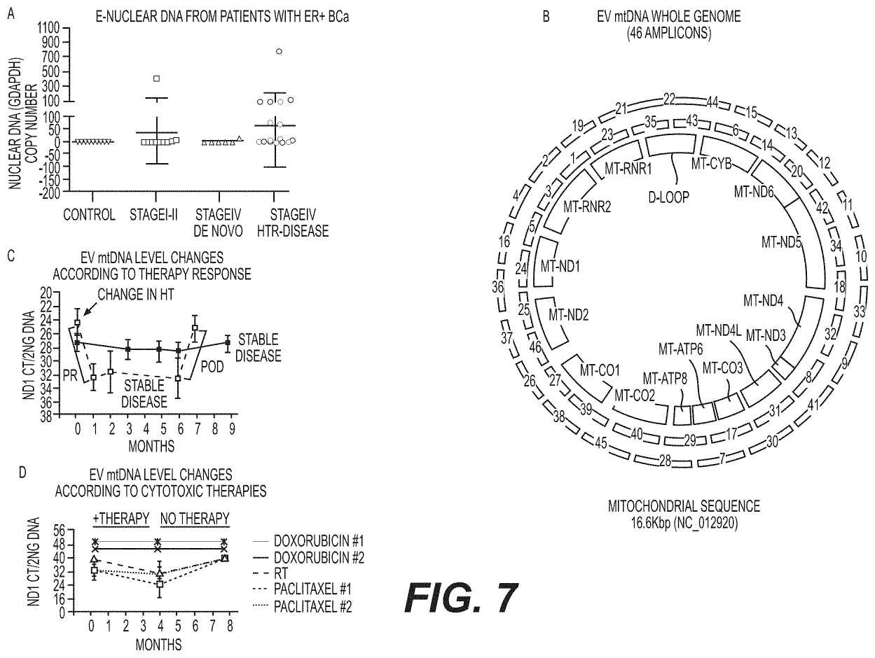

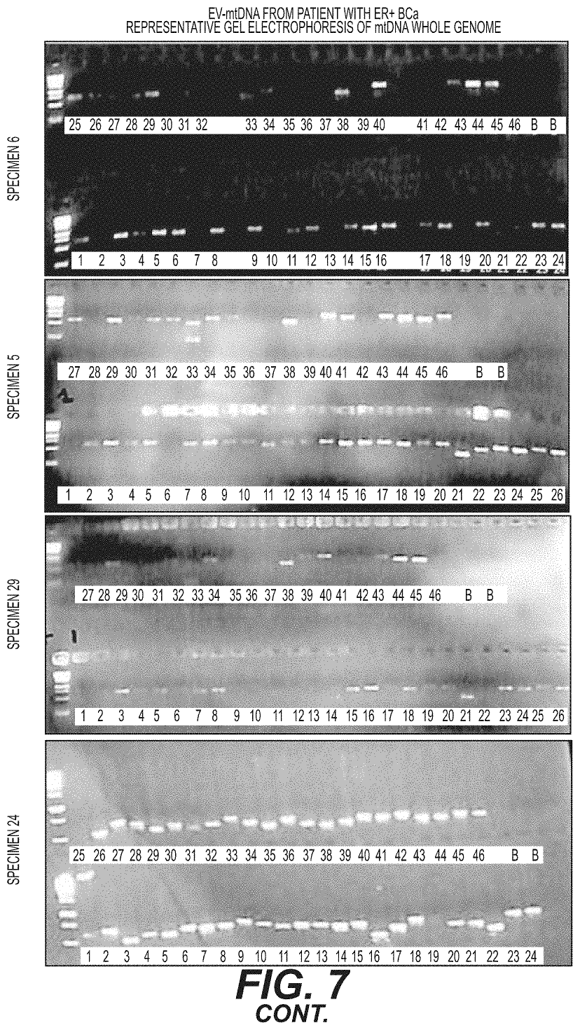

In one embodiment, the present application provides a method for reducing the likelihood of development of resistance to hormonal-therapy in patients with metastatic breast cancer. The method comprises the step of administering to a patient in need of such treatment an effective amount of an agent that inhibits horizontal transfer of mtDNA. In one embodiment, the method comprises the steps of isolating extracellular vesicles (EVs) from a plasma sample obtained from a breast cancer patient by centrifugation and ultracentrifugation and/or filtration, isolating DNA from isolated EVs, and determining the amount (copy number by semi quantitative PCR) of mtDNA ND1 DNA (and others genes including ND2, COX2, COX1, ATP8, ATP6, COX3, ND3, ND4L, ND4, NDS, ND6, CytB) in the isolated nucleic acid, wherein the presence of mtDNA ND1 gene (or other mtDNA genes) above a threshold level of ≥100 fold change of a non HTR subject (copy number; see Table 5) is indicative of a likelihood of developing HTR metastatic breast cancer in the patient (FIGS. 1 and 7: the biogenesis of mtDNA high EVs from the plasma of patients and in experimental breast cancer models). We demonstrated that the administration of mtDNA low EVs (FIG. 4-5) is a way to reduce the likelihood of resistance to hormonal therapy in vitro and in vivo.

Method for Diagnosing DNA Damage In Vivo: mtDNA High EVs in the Circulation of Patients

It is well known that DNA-damaging drugs such as Doxorubicin induce cardiac toxicity and heart failure through mitochondrial damage. The EV-derived mtDNA copy number with therapy may function as a marker of future cardiac toxicity. It was observed that ND1 DNA level was higher in EVs from patients receiving therapies which damage DNA (e.g. Doxorubicin and Radiation therapy) but not in those receiving non-genotoxic drugs (e.g. Paclitaxel) (FIG. 7, Panel d). Notably, once these therapies were finished resolution of mtDNA high EVs was observed (FIG. 7, Panel d). Thus, the presence of mitochondrial DNA in exosomes could not only predict the development of resistance to endocrine therapy but also as an acute marker of DNA damage.

Equally important, this application provides a method for providing or rescuing metabolic disorders whereby patients have diseases due to mtDNA mutations or deficiencies which can be rescued by administering to a patient EVs laden with wild type or a specific mtDNA variant which is required to rescue or complement mitochondrial activity.

As results of the following methods, another important aspect of the present application relates to a kit for detecting mtDNA related conditions in a subject. In one embodiment, the kit comprises one or more reagents for isolating EVs from a biological sample, one or more reagents for isolating DNA from isolated EVs, and one or more reagents for detecting one or more mtDNA markers in isolated DNA. In some embodiments, the kit comprises one or more DNA primers listed herein (see Primer Tables (Tables 1-4)). In some embodiments, the kit further comprises one or more DNA primers listed in Primer Tables. In some embodiments, the kit further comprises a filter or beads for isolating EVs from a body fluid. In some embodiments, the one or more mtDNA markers comprise ND1 gene.

Method for Delivering mtDNA to Patients with a Condition Related to mtDNA Damage or Deficiency.

Another aspect of the present application relates to a method for enhancing or rescuing a medical condition related to mtDNA damage or deficiency. These conditions include cardiac disease, metabolic deficiencies and neurological conditions (e.g. Kearns-Sayre syndrome (KSS), Leber Hereditary Optic Neuropathy (LHON), subacute sclerosing encephalopathy, progressive external ophthalmoplegia, Pearson Syndrome, Leigh syndrome, exercise-induced muscle pain, fatigue and rhabdomyolysis, amino-glycoside-induced hearing loss, mitochondrial myopathy, encephalopathy, lactic acidosis and stroke-like episodes (MELAS), biventricular cardiac hypertrophy, diabetes mellitus and deafness, strokes and migraine and seizures and ataxia, retinitis pigmentosa, and ptosis (NARP), lactic acidaemia in children, myoclonic epilepsy with ragged red fibers that is associated with ragged-red fibres, Alzheimer disease, Parkinson disease, myoneurogenic gastrointestinal encephalopathy (MNGIE) and others (see, e.g., Nat Rev Genet. 2005 May; 6(5):389-402; Walker and Chalkia, Cold Spring Harb Perspect Biol. 2013 Nov. 1; 5(11): a021220. doi: 10.1101/cshperspect.a021220). Briefly, wild type mtDNA either isolated from the patient subject or laboratory derived (recombinant mtDNA) can be packaged in EVs previously isolated from the same patient subject (plasma) and then re-injected in the circulation of the subject.

This method includes: 1. diagnosing a mtDNA related disorder in a subject (mtDNA level and genetic variants in the subject's EVs and tissue); 2. Isolating EVs from the subject; 3. Generating laboratory EVs loaded with mtDNA (wild type or a specific variant which is required to rescue or complement mitochondrial activity) by genetic insertion of recombinant mtDNA in subject's EVs; 4. administration of these functional mtDNA high EVs in the subject either in the circulation (plasma) and/or locally (within affected tissue such as liver, muscle or brain).

In one embodiment, this method is supported by the data showing that mtDNA high EVs promoted hormonal therapy resistance via the generation of OXPHOS proficient cancer cells in vitro and in vivo (FIGS. 4 and 5). These data suggest EVs can rescue metabolic disorders in patients with diseases due to mtDNA mutations or deficiencies (loss of) via the transfer of EVs laden with wild type or a specific mtDNA variants which are required to rescue or complement mitochondrial activity.

Method for Enhancing the Biogenesis of mtDNA High EVs

Another important aspect of the present application relates to a method to increase the biogenesis of mtDNA high EVs. Such a method includes the exposure of normal and cancer cells to hypoxia-reoxygenation culture condition (FIG. 14, Panel a) and chemo/radio therapy (FIG. 7, Panel d). In one embodiment, the method comprises one or more reagents for isolating EVs from cells, one or more reagents for isolating DNA from isolated EVs, and one or more reagents for detecting one or more mtDNA markers in isolated DNA (FIG. 14).

### Kits

As results of the above-described methods, another important aspect of the present application relates to a kit for detecting a mtDNA related conditions in a subject. In one embodiment, the kit comprises one or more reagents for isolating EVs from a biological sample, one or more reagents for isolating DNA from isolated EVs, and one or more reagents for detecting one or more mtDNA markers in isolated DNA. In some embodiments, the kit comprises one or more DNA primers listed in Tables 1-4. In some embodiments, the kit further comprises one or more DNA primers listed in Primer Tables 2 and 3. In some embodiments, the kit further comprises a filter or beads for isolating EVs from a body fluid. In some embodiments, the one or more mtDNA markers comprise ND1 gene.

### EXAMPLES

### Example 1

### Materials and Methods

Patient Plasma Collection

Human peripheral plasma samples (5-10 mls) were obtained in EDTA tubes from 1) control healthy subjects, 2) patients with early stage breast cancer (who had their tumor surgically removed) and were cancer free, 3) de novo metastatic disease who had not started therapy and 4) in those with progression of metastatic disease on anti-estrogen therapy at Memorial Sloan Kettering Cancer Center and all pathologically confirmed (Table 5). All individuals provided informed consent for blood donation on approved institutional protocols (MSKCC IRB 12-137). Once blood was drawn, plasma was isolated within 4 hours. No samples were frozen.

### Isolation and Characterization of EVs

Extracellular vesicles isolation from the plasma of patients and the conditioned media of cancer and stromal cell cultures was performed using sequential centrifugation as previously described (Peinado, H. et al. Melanoma exosomes educate bone marrow progenitor cells toward a pro-metastatic phenotype through MET. Nature medicine 18, 883-891 (2012)). Briefly plasma and conditioned media was centrifuged at 3000 g for 20 min to remove any cell contamination. To remove apoptotic bodies, mitochondrial particles and large cell debris, the supernatants were centrifuged at 12,000 g for 30 min. EVs were collected by spinning at 100,000×g for 70 min. EVs were resuspended in 25 ml of 1× PBS and loaded on a 5 ml 30% sucrose cushion (300 g/L sucrose, 24 g/L Tris base, pH 7.4). Samples were centrifuged at 100,000×g for 90′ at 4° C. 3.5 ml of the cushion, containing EVs, was diluted with 1× PBS and centrifuged at 100,000×g for 90′ at 4° C. The EV containing pellet was resuspended in 25 μl of PBS. The EVs were treated or not, depending on the experiment, with 1U of Baseline-ZERO™ DNase0 solution (Epicentre®) for 1 h at 37° C., in order to digest DNA adherent to the surface of EVs or present in solution and subsequently inactivated for 10′ at 65° C. Vesicle preparations were verified by electron microscopy. Exosome size and particle number were analyzed using the LM10 or DS500 nanoparticle characterization system (NanoSight, Malvern Instruments) equipped with a blue laser (405 nm).

### Cell Lines and Primary Cultures

Human cancer cell lines (Hela, Caski -cervical carcinoma-), human breast cancer cell lines (MCF7, ZR751, T47D, and BT474), human bone marrow stromal cell lines (HS5, HS27a) and human normal fibroblasts (MRC5, HMF) were purchased from the American Type Culture Collection (ATCC). Murine CAFs (mCAFs) were isolated from xenografts by FACS purification (GFP negative, EpCAM negative). All cells were mycoplasma free and maintained in MEM and RPMI (ATCC and MSKCC Media Core) supplemented with 5% fetal bovine serum (Media Core), 2 mM glutamine, 100 units ml-1 penicillin, and 0.1 mg ml-1 streptomycin (Media Core).

Primary cultures from breast cancer xenografts were established from primary and metastatic tissues of xenografts treated with and without HT. Cancer cells were obtained following tissue enzymatic digestion (Coll agenase/Hyaluronidase), cell sorting purification (GFP+/DAPI−) and passaged in vitro. Fulvestrant (HT) was purchased from Sigma (Sigma-Aldrich). For FACS/Flow analyses xenograft derived tumors and metastases were digested in sterile Epicult media (Stem Cell Technology), minced and incubated for 3 hours in the presence of Collagenase/Hyaluronidase (1,000 Units/sample). Cells were washed with PBS supplemented with 1% bovine serum albumin (PBS-BSA 1%) and filtered through a 40 μM nylon mesh (BD Biosciences). For the detection of EpCAM antigen, cells were stained in a volume of 100 μl (PBS-BSA 1%) with EpCAM-FITC antibody (250 ng/106 to-108 cells, Clone VU-1D9, Stem Cell Technologies). Cells were labeled on ice for 30 min and analyzed (BD FACS Aria III, Flow Core). For flow plot analyses, samples were run using FlowJo 7.5 software (Tree Star). For all extracellular vesicle studies, cells were grown in exosome-depleted FBS (Gibco, Thermo Fisher Scientific).

Primary human CAFs were isolated from patients with resected bone metastasis (MSKCC, IRB #97-094). Freshly isolated metastasis were minced and incubated at 37° C. for 8-12 h with Collagenase/Hyaluronidase enzyme mix (1,000 Units, Voden Medical), washed and cultured in Epicult media (Voden Medical). Mammosphere-depleted supernatants were centrifuged at 450×g for 10 min; stromal cell containing pellets were further enriched by negative selection (EpCAM negative) and expanded/maintained in DMEM with 5% FBS for n=10 passages.

Mitochondrial depleted cells (ρ0 cells) were generated by administering CAFs/cancer cultures with ρ0 media (2 mM glucose, 1% fetal calf serum, 0.4 μg/ml ethidium bromide, 50 μg/mluridine, 100 μg/ml pyruvate) for 2 months.

### In Vivo EV Education of Hormonal Therapy Resistant Experimental Breast Cancer

All cancer cell lines were engineered to express a GFP positive luciferase expression vector for in vitro and in vivo imaging studies. Prior to in vivo inoculation, cancer cells were FACS sorted (for GFP) and injected bilaterally in the mammary fat pads of 5-7 weeks old non-obese diabetic/severe combined immunodeficiency mice (NOD/SCID, obtained from NCI Frederick, Md.). For each in vivo experiment, cancer cells were mixed with an equal volume of Matrigel™ (BD Biosciences) in a total volume of 50 μl. Bioluminescence (BLI: Xenogen, Ivis System) was used to monitor both tumor growth (weekly) and metastatic burden (at necropsy). Pre-clinical therapeutic trials were generated using xenografts from tumorigenic MCF7 and ZR751 clones treated with tamoxifen pellet as previously described (Sansone, P. et al. Self-renewal of CD133(hi) cells by IL6/Notch3 signaling regulates endocrine resistance in metastatic breast cancer. Nature communications 7, 10442 (2016)) or fulvestrant (Faslodex, HT, AstraZeneca), which was given intra-muscularly in the posterior/popliteal muscles (100 mg/injection/weekly). For metastatic assays, primary tumors were removed from mice bearing MCF7 xenografts and metastatic progression was followed by BLI in the presence of tamoxifen (pellet). All the surgical procedures and animal care followed the institutional guidelines and an approved protocol from the IACUC at MSKCC. The in vivo role of mtDNA+CAF-EVs in the promotion of HTR disease (FIG. 4d) was determined by first injecting HT-naïve cells (105 MCF7/ZR751) into the MFPs of NOD/SCID mice followed by the injection of 3×109 CAF-EVs (mtDNAhi-EV or mtDNAlo-EVs; n=5 mice/group) into the venous circulation (retro-orbital injection, 3×10⁹ particles/mouse/weekly) of mice. See FIG. 4/5, description of cell lines and EV isolation described above. EV-number was determined by Nanosight. After 8 weeks, once tumors were established (˜1 cm, BLI 2×109), HT (fulvestrant, 100 mg/mouse/weekly) was administered for an additional 6 weeks. The in vivo role of CAF-EVs in the promotion of hormonal therapy resistant luminal breast cancer was determined by injecting CAF-EV (Mu-CAFs, isolated from hormonal therapy resistant xenografts and cultured in vitro) and mitochondrial DNA depleted EVs (from mtDNA depleted CAFs) into the venous circulation (retro-orbital injection, 3×10⁹ particles/mouse/weekly) of tumor-bearing mice.

### Mitochondrial Activity Assays

Mitochondrial respiration and OXPHOS potential in cells was assessed using Seahorse technology (www.seahorsebio.com)(Qian, W. & Van Houten, B. Alterations in bioenergetics due to changes in mitochondrial DNA copy number. Methods 51, 452-457 (2010)). Oxygen consumption rate (OCR), extracellular acidification rate and glycolytic potential were determined using the Seahorse extracellular flux analyzer (XF-96, XF-24 Seahorse Bioscience). To allow comparison between experiments, data are presented as OCR (pMol/min). Graphs showing the kinetic of OCR under basal condition followed by the sequential addition of oligomycin (1 μM), 2DG (50 mM), rotenone (300 nM) and glucose (20 mM) are reported (cell mito stress kit from seahorse bioscience). The progress curve is annotated to show the relative contribution of basal, ATP-linked (oligomycin) oxygen consumption and the reserve capacity of the cells (after the addition of 2DG+rotenone). Furthermore OXPHOS potential was determined by measuring the area of the progress curve after the addition of glucose and rotenone/oligomycin.

For in vitro labeling of mitochondria, MitoTracker dye was used (Invitrogen). For mitochondrial membrane potential TMRE was used according to manufacturer's protocol (Abcam, ab113852).

To measure mitochondrial complex activity, cell pellets were resuspended in assay buffer (125 mM sucrose, 0.1 mM EDTA, 25 mM Tris-HCl pH 7.5, 0.025% Lauryl-β-D-maltoside), and enzymatic activities were assayed with 0.1-0.4·106 cells/ml at 25° C. spectrophotometrically. NADH:HAR reductase activity of Complex I was measured as a decrease in absorption at 340 nm (ε340 nm=6.22 mM-1 cm-1) with 200 μM NADH, 1mM HAR and 1 mM KCN. Complex IV activity was measured as cytochrome oxidation at 550 nm (ε550 nm=21.5 mM-cm-) with 50 μM ferrocytochrome c. Activities are expressed in μmol of substrate.min-1.mg of protein-1. For protein measurement cells were pelleted and resuspended in 1% deoxycholate. Protein content was determined with a BCA assay.

Exit from Dormancy via CAF-EVs

Generation of HTD cells was performed by treating ER+(GFP+) breast cancer cell lines (MCF7, ZR751, BT474) with HT (fulvestrant, 10 μM) for 2 months. Single, non-proliferating cancer cells were FACS purified by gating on GFP+ cells and by DAPI exclusion staining by flow cytometry (Dako Cytomation). Viable cell number was subsequently determined using trypan blue exclusion and cell counting using bright field microscopy HTD cells were then cultured in vitro or injected in vivo for further studies. In vitro assays: HTD cell were cultured in 96-well dishes (1000 cells/well) and treated with 107 EVs (mtDNAhi-EV or mtDNAlo-EVs) weekly (4×). Half of the cultures were also treated with HT (fulvestrant, 10 μM, Sigma-Aldrich). Proliferation was determined by Cal cein AM technique (InVitrogen). HTD were cultured in low attachment (Corning) 24-well plates. These were treated with 3×109 mCAF-derived EVs (wt-mtDNAhi or ρ0-mtDNAlo) weekly ×4 in the presence with HT (fulvestrant, 10 μM/weekly). After 40 days, mammosphere number (bright field microscopy) was determined after 40 days. In vivo assays: Dormant ZR751 cells (HTD) were isolated and 105 cells were injected in the IV inguinal MFP of NOD/SCID mice. All mice were subsequently injected with 3×109 mCAF-derived EVs (wt-mtDNAhi or ρ0-mtDNAlo) weekly ×8 (n=5 mice/group). Tumor growth was determined by BLI and after necropsy.

### Electron Microscopy (EM)

Cells were washed with serum-free media or appropriate buffer. Both EVs and cells were fixed with a modified Karmovsky's fix of 2.5% glutaraldehyde, 4% paraformaldehyde and 0.02% picric acid in 0.1M sodium caocdylate buffer at pH 7.2. Following a secondary fixation in 1% osmium tetroxide, 1.5% potassium ferricyanide, samples were dehydrated through a graded ethanol series and embedded in an epon analog resin. Ultrathin sections were cut using a Fiatome diamond knife (Diatome, USA, Hatfield, Pa.) on a Leica Ultracut S ultramicrotome (Leica, Vienna, Austria). Sections were collected on copper grids and further contrasted with lead citrate and viewed on a JEM 1400 electron microscope (JEOL, USA, Inc., Peabody, MA) operated at 120 kV. Images were recorded with a Veleta 2K×2K digital camera (Olympus-SIS, Germany).

### Nucleic Acid Extraction

DNA extraction: cell pellets and EVs (ultracentrifugation of 1012 cells) were resuspended in 25 μl of 1× PBS followed by the addition of 450 μl of DNA extraction buffer (SDS 0.5-1%, Tris-HCl 50 mM pH 8.0, EDTA 0.1 M) and 0,1 mg/ml proteinase K 20 mg/ml (ThermoFisher Scientific) and incubated O/N at 56° C. 500 μl of phenol/chloroform (ThermoFisher Scientific) was added to each sample and centrifuged at 13,000 rpm for 5′ at room temperature. The upper phase, containing the DNA was transferred to a new tube where 500 μl of chloroform were added. Samples were centrifuged at 13,000 rpm for 5′ at room temperature; the DNA was washed a second time by repeating this step. The upper phase was transferred to a new tube with 450 μl of isopropanol and 50 μl of NaAc 3M. The samples were centrifuged at 13,000 rpm for 10′ at 4° C. The supernatant was discarded and the pellet washed with 750 μl 70% EtOH and centrifuged at 13,000 rpm for 5′ at 4° C. The DNA pellet was air dried, resuspended in 20 μl A of DEPC H2O and incubated at 37° C. for 30′. DNA concentration was measured by loading 1 μl of DNA on a Thermo Scientific NanoDrop™ 1000 Spectrophotometer and stored at −20° C. until further analyzed. For RNA extraction we used trizol (Invitrogen). EVs were added with 500 μl of Trizol and mixed. The samples were centrifuged at 12,000×g for 30 seconds. 200 μl of chloroform were added, mixed by inversion and incubated for 2-3′ at RT. After a centrifugation at 12,000×g for 15′ at 4° C., the upper phase was transferred to a new tube. 400 μl of isopropanol and 3 μl of glycogen were added to the sample and incubated O/N at −20° C.; the samples were then centrifuged at 12,000×g for 10′ at 4° C. The supernatant was discarded and the pellet washed in 750 μl of cold 75% EtOH. The RNA was pelleted with a centrifugation at 8,000×g for 20′ at 4° C., air dried and resuspended in 10 μl of DEPC H2O. The RNA concentration was measured with a Thermo Scientific NanoDrop™ 1000 Spectrophotometer and treated for 1 hour at 37° C. with 1 U (for EVs) or 2 U (for cells) of Baseline-ZERO™ DNase I (Epicentre®). RNA was stored at −80° C.

### MtDNA/nDNA Copy Number Quantification

mtDNA and nDNA were amplified by standard PCR (mitochondrial ND1, ND5; nuclear GAPDH, Actin: both human and murine, see primer list at the end of the methods section) and subsequently extracted from agarose gels using the Nucleospin®Gel and PCR clean-up kit (Macherey Nagel) and quantified using Agilent 2100 Bioanalyzer Instrument. ND1, ND5, GAPDH and Actin copy number was calculated using the following formula: Copy number-(Ng ×6.022×1023)/(Length×1×109×650): ng-concentration of the eluted sample, 6.022×1023=Avogadro's number, Length=length of amplicon in bps, 1×109×650=average weight of a base pair in ng. Standard curves were created by qPCR amplifying serial dilutions of the amplicon of interest and used to interpolate the CT data for quantification.

Whole mtDNA Amplification and Sequencing

Total DNA (1-5 ng) was used for mtDNA amplification with the MitoALL Resequencing kit. PCR amplification and Sanger sequencing of 46 amplicons was performed as previously described (Kurelac, I. et al. Somatic complex I disruptive mitochondrial DNA mutations are modifiers of tumorigenesis that correlate with low genomic instability in pituitary adenomas. Human molecular genetics 22, 226-238 (2013)). Electropherograms were analyzed by SeqScape. Functional annotation was performed by applying previously described methods (Santorsola, M. et al. A multi-parametric workflow for the prioritization of mitochondrial DNA variants of clinical interest. Human genetics 135, 121-136 (2016)) and consulting Mitomap (Lott, M.T. et al. mtDNA Variation and Analysis Using Mitomap and Mitomaster. Current protocols in bioinformatics/editoral board, Andreas D. Baxevanis [et al.] 44, 1 23 21-26 (2013)) and HmtDB (Rubino, F. et al. HmtDB, a genomic resource for mitochondrion-based human variability studies. Nucleic acids research 40, D1150-1159 (2012)). Murine mtDNA was also sequenced using a specific 46-amplicon PCR technique (this method is under patent approval and its details cannot be included).

### Reverse Transcription PCR (RT-PCR) and Microarray Analyses

cDNA was obtained by retro transcribing 1 μg of RNA-previously treated with 1 U (EVs) or 2 U (cells) of Baseline-ZERO™ DNase0 (Epicentre®)- and using iScript™Select cDNA synthesis Kit (Bio-Rad). The cDNA was kept at −80° C. for further analysis. For microarray analysis 250 ng of RNA was processed, assayed and run on Illumina GX Human HT12 platform according to the manufacture's' protocol at the Genomic Molecular Core Facility (MSKCC). Data were analyzed, submitted at GEO (GSE84104) and a table with fold change was generated.

### Real Time PCR

DNA and cDNAs were amplified by quantitative PCR (qPCR) using the Applied Biosystem Viia™ 7 Real-Time PCR System in the Power SYBR® Green PCR Master Mix Buffer. Each sample was run in triplicate. DNA amplification was performed on 2 ng DNA/reaction; cDNA amplification was performed on 1 μl of the cDNA/triplicate. All primers used in the Real Time assay are listed at the end of the section. For analysis, ΔΔ_{ct} method was applied and fold change was calculated (2^{-ΔΔct}). In order to verify the specificity of the amplicons, other than the analysis of the Melting Temperature, amplicons were visualized on a 2% agarose gel using the ChemiDoc™ XRS+System (Bio-Rad).

Standard PCR on DNA and cDNA

DNA was isolated using phenol/chloroform (ThermoFi sher Scientific). Each amplification reaction was performed on a total of 2 ng of DNA using the GeneAmp® PCR System 9700, version 2.5. The amplification program was the following: (i) Polymerase activation (2 min at 95° C.), (ii) amplification stage (35 cycles, with each cycle consisting of 30 seconds at 95° C., 30 seconds at 60° C., and 60 seconds at 72° C.), and (iii) extension stage (5 min at 72° C.). All amplification reactions were performed using the GoTaq®Flexi DNA Polymerase kit (Promega). PCR products were resolved on a 2% agarose gel. All primers used for this assay are listed at the end of the methods section.

Long Range mtDNA PCR

Long range PCR was used in order to amplify the whole murine mitochondrial DNA using 3 pairs of overlapping primers (Dames, S., Eilbeck, K. & Mao, R. A high-throughput next-generation sequencing assay for the mitochondrial genome. Methods in molecular biology 1264, 77-88 (2015)). The 5′ extremity of the primers was modified with an aminoC6 sequence so that the annealing temperature can reach 68° C. and lead to a more specific amplification. Each amplification reaction was performed on a total of 20 ng of DNA with a GeneAmp®PCR System 9700 version 2.5 model. Amplicons were visualized on a 0.8% agarose gel using the ChemiDoc™ XRS+System (Bio-Rad).

### EV Labeling and Transfer to Recipient Cells

EVs from CAFs, were labeled using the PKH67 Green Fluorescent Cell Linker Kit for General Cell Membrane Labeling (Sigma-Aldrich). 105 HTD cells (MCF7 cells treated with HT-see description of cell lines) were grown in Nunc®Lab-Tek®Chamber Slide (Sigma-Aldrich), previously coated with fibronectin to allow for cell adhesion. Cells were treated with 3×108 labeled EVs and their localization determined 48 hours later. Mitochondria were labeled using Red-MitoTracker® (25 nM for 30 minutes at 37° C.). Cells were washed and fixed (4% paraformaldehyde) and nuclei were stained with DAPI. Fluorescent confocal microscopy (Nikon Eclipse TE2000U) was used to localize EVs (green channel-PKH67), mitochondria (red channel) and nuclei acid (far red EtBr) and analyzed using Nikon software (EZ-C1 3.6).

### Nuclease Treatment of EVs

RNA/DNA isolated from EVs was extracted, treated with 1 U of Baseline-ZERO™DNase0 (Epicentre), in order to eliminate contaminating ss- and ds-DNA, and processed with different nucleases to analyze its chemical and physical status. Enzymes were heat inactivated with an incubation of 10 minutes at 70° C.

### Protein and In Vitro Studies

For immunoblotting assays, cells were lysed in buffer (50 mmol/L Tris at pH 7.5, 150 mmol/L NaCl, 5 μg/mL aprotinin, pepstatin, 1% NP-40, lmmol/L EDTA, 0.25% deoxycholate, and protease inhibitor cocktail tablet, Sigma). Proteins were separated by SDS-PAGE, transferred to PVDF membranes and blotted with specific antibodies: OXPHOS complex Antibody (which recognizes also ATP5A1 protein) (Invitrogen, 45-7999); tubulin (Santa Cruz Biotech. Inc: 10D8); CD63 (EXOAB-CD63A-1, System Bioscience) and Alix (3A9-sc-53538, Santa Cruz Biotech. Inc). For immunostaining assays: organs were collected and fixed overnight in 4% paraformaldehyde, washed, embedded in paraffin and sectioned (Histo-Serve). H&E staining was performed by standard methods. The enrichment of CAFs in xenografts was determined by desmin immunohistochemistry (IHC) in tumor-derived sections. IHC was performed on Leica Bond RX (Leica Biosystems) with 1 μg/ml Desmin Rabbit polyclonal antibody (Abcam cat#ab8592). For determination of cell viability, we seeded 2,500 cells per well in 96-well plates and treated them with fulvestrant (10 μM). Viable cells were determined 7-14 days after treatment using trypan blue and cell counting using bright field microscopy or DAPI exclusion staining by flow cytometry (Dako Cytomation). Proliferation assays were carried out using CalceinAM technology (Invitrogen) or bioluminescence: cells were seeded in 96 wells plates treated with the pre-fluorescent/luciferin compound for 20 min and fluorescence was read using a plate reader (SpectraMax plate platform/IVIS BLI xenogen).

Semi-Quantitative Mass Spectrometry analysis of CAF EV 100821 Mass spectrometry analyses of EV were performed at the Rockefeller University Proteomics Resource Center (New York, N.Y., USA) using 10 μg of CAF-EV protein as previously described (Hoshino, A. et al. Tumor exosome integrins determine organotropic metastasis. Nature 527, 329-335 (2015)). Samples were denatured using 8 M urea, reduced using 10 mM dithiothreitol, and alkylated using 100 mM iodoacetamide, followed by proteolytic digestion with endoproteinase LysC (Wako Chemicals), and subsequent digestion with trypsin (Promega) for 5 h at 37° C. and quenched with formic acid and the resulting peptide mixtures were desalted. Samples were dried and solubilized in buffer containing 2% acetonitrile and 2% formic acid. Approximately 3-5 μg of each sample was analyzed by reverse-phase nano-LC-MS/MS (Ultimate 3000 coupled to QExactive, Thermo Scientific). Following loading on the C18 trap column (5 μm beads, Thermo Scientific) at a flow rate of 3 μl min-1, peptides were separated using a 75-μm-inner-diameter C18 column (3 μm beads Nikkyo Technos) at a flow rate of 200 nl min-1, with a gradient increasing from 5% Buffer B (0.1% formic acid in acetonitrile)/95% Buffer A (0.1% formic acid) to 40% Buffer B/60% Buffer A, over 140 min. All LC-MS/MS experiments were performed in data-dependent mode. Precursor mass spectra were recorded in a 300-1,400 m/z mass range at 70,000 resolution, and 17,500 resolution for fragment ions (lowest mass: m/z 100). Data were recorded in profile mode. Up to 20 precursors per cycle were selected for fragmentation and dynamic exclusion was set to 45 s. Normalized collision energy was set to 27. Data were extracted and searched against Uniprot complete Mouse proteome databases (January 2013) concatenated with common contaminants using Proteome Discoverer 1.4 (Thermo Scientific) and Mascot 2.4 (Matrix Science). All cysteines were considered alkylated with acetamide. Amino-terminal glutamate to pyroglutamate conversion, oxidation of methionine, and protein N-terminal acetylation were allowed as variable modifications. Data were first searched using fully tryptic constraints. Matched peptides were filtered using a Percolator-based 1% false discovery rate. Spectra not being matched at a false discovery rate of 1% or better were re-searched allowing for semi-tryptic peptides. The average area of the three most abundant peptides for a matched protein was used to gauge protein amounts within and in between samples.

### Statistical Analysis

Statistical analysis was performed by SPSS (SPSS Incorporation). Continuous variables were analyzed by unequal variance t-test, paired t-test (for samples, n=2), general linear model (GLM) Anova or GLM for repeated measures (samples, n>2). Mann-Whitney, Wilcoxon and Friedman tests were used to analyze ordinal variables. P values were adjusted for multiple comparisons according to Bonferroni correction. All the tests were two-sided. P<0.05 was considered significant.

### Primer Tables

The reference sequences used for primers design are NC_010339 for the murine mitochondrial DNA/RNA, NC_012920 for the Human mitochondrial DNA/RNA.

TABLE 1
Murine Mitochondrial Primers
Murine Mitochondrial DNA

| | | | Length | | |
|---|---|---|---|---|---|
| Gene | Sequence 5′-3′ | Position | (bps) | Assay | Reference |
| DLoop | AGGTTTGGTCCTGGCCTTAT (SEQ ID NO: 1) | 72 F | 149 | Real | SnapGene |
| | GTGGCTAGGCAAGGTGTCTT (SEQ ID NO: 2) | 221 R | | Time/PCR | Viewer Software |
| ND1 (1)⁹ | CTAGAAACCCCGAACCAAA (SEQ ID NO: 3) | 1323 F | 105 | | |
| | CCAGCTATCACCAAGCTCGT (SEQ ID NO: 4) | 1428 R | | | |
| ND1 (2) | CAGCCGGCCCATTCGCGTTA (SEQ ID NO: 5) | 3398 F | 197 | | |
| | AGCGGAAGCGTGGATAGGATGC (SEQ ID NO: 6) | 3595 R | | | |
| ND2 | TCCTCCTGGCCATCGTACTCAACT (SEQ ID NO: 7) | 4123 F | 131 | | |
| | AGAAGTGGAATGGGGCGAGGC (SEQ ID NO: 8) | 4254 R | | | |
| COX1 | CCAGTGCTAGCCGCAGGCAT (SEQ ID NO: 9) | 5927 F | 127 | | |
| | TCTGGGTGCCCAAAGAATCAGAACA (SEQ ID NO: 10) | 6054 R | | | |
| COX2 | AGTTGATAACCGAGTCGTTCTGCCA (SEQ ID NO: 11) | 7425 F | 123 | | |
| | TCGGCCTGGGATGGCATCAGT (SEQ ID NO: 12) | 7548 R | | | |
| ATP8 | ATGCCACAACTAGATACATCAACA (SEQ ID NO: 13) | 7768 F | 176 | | |
| | GGGGTAATGAATGAGGCAAA (SEQ ID NO: 14) | 7944 R | | | |
| ATP6 | GCTCTCACTCGCCCACTTCCTTCC (SEQ ID NO: 15) | 8297 F | 529 | | |
| | GCCGGACTGCTAATGCCATTGGTT (SEQ ID NO: 16) | 8826 R | | | |
| COX3 | ACCTACCAAGGCCACCACACTCC (SEQ 1D NO: 17) | 8804 F | 149 | | |
| | GCAGCCTCCTAGATCATGTGTTGGT (SEQ ID NO: 18) | 8953 R | | | |
| ND3 | ACCCTACAAGCTCTGCACGCC (SEQ ID NO: 19) | 9585 F | 385 | | |
| | GCTCATGGTAGTGAAGTAGAAGGGCA (SEQ ID NO: 20) | 9970 R | | | |
| ND4 L | TCGCTCCCACCTAATATCCACATTGC (SEQ ID NO: 21) | 9945 F | 141 | | |
| | GCAGGCTGCGAAAACCAAGATGG (SEQ ID NO: 22) | 10086 R | | | |
| ND4 | TCGCCTACTCCTCAGTTAGCCACA (SEQ ID NO: 23) | 11026 F | 115 | | |
| | TGATGATGTGAGGCCATGTGCGA (SEQ ID NO: 24) | 11141 R | | | |
| ND5 | TCGGAAGCCTCGCCCTCACA (SEQ ID NO: 25) | 12868 F | 105 | | |
| | AGTAGGGCTCAGGCGTTGGTGT (SEQ ID NO: 26) | 12973 R | | | |
| ND6 | AATACCCGCAAACAAAGATCACCCAG (SEQ ID NO: 27) | 13585 F | 99 | | |
| | TGTTGGGGTTATGTTAGAGGGAGGGA (SEQ ID NO: 28) | 13684 R | | | |
| Cytb | ACAGCAAACGGAGCCTCAA (SEQ ID NO: 29) | 14394 F | 134 | | |
| | TGCTGTGGCTATGACTGCGAACA (SEQ ID NO: 30) | 14528 R | | | |

TABLE 2
Human Mitochondrial Primers
Human Mitochondrial DNA-RNA

| | | | Length | | |
|---|---|---|---|---|---|
| Gene | Sequence 5′-3′ | Position | (bps) | Assay | Reference |
| DLoop | TGGCCACAGCACTTAAACACATCTC (SEQ ID NO: 31) | 321 F | Real | Real | SnapGene |
| | GGGTTGTATTGATGAGATTAGTAGTATGGGAG | 496 R | 175 | Time/ | Viewer |
| | (SEQ ID NO: 32) | | | PCR | Software |
| 12S | CCCGTTCCAGTGAGTTCACCC (SEQ ID NO: 33) | 706 F | 112 | | |
| | TGTGGGGGTGCCCTTTGTC (SEQ ID NO: 34) | 818 R | | | |
| 16S | AACTTTGCAAGGAGAGCCAAAGC (SEQ ID NO: 35) | 1873 F | 205 | | |
| | GGGATTTAGAGGGTTCTGTGGGC (SEQ ID NO: 36) | 2078 R | | | |
| ND1 | ACGCCATAAAACTCTTCACCAAAG (SEQ ID NO: 37) | 3458 F | 103 | Real | |
| | TAGTAGAAGAGCGATGGTGAGAGCTA (SEQ ID NO: 38) | 3561 R | | Time/ | |
| | | | | PCR | |
| ND2 | CTTCTGAGTCCCAGAGGTTACCCA (SEQ ID NO: 39) | 4805 F | 182 | Real | |
| | CCGTCAACTCCACCTAATTTGGTTTG (SEQ ID NO: 40) | 4987 R | | Time/ | |
| COX1 | TGCCATAACCCAATACCAAACGC (SEQ ID NO: 41) | 6425 F | 112 | PCR | |
| | CTGTTAGTAGTATAGTGATGCCAGCAGCTAGG | 6537 R | | | |
| | (SEQ ID NO: 42) | | | | |
| COX2 | CTACGGTCAATGCTCTGAAATCTGTG (SEQ ID NO: 43) | 8161 F | 153 | | |
| | GCTAAGTTAGCTTTACAGTGGGCTCTAG (SEQ ID NO: 44) | 8314 R | | | |
| ATP6 | GAAAATCTGTTCGCTTCATTCATTGCC (SEQ ID NO: 45) | 8533 F | 138 | | |
| | GCTGATTAGTGGTGGGTTGTTACTG (SEQ ID NO: 46) | 8671 R | | | |
| COX3 | CGATACGGGATAATCCTATTTATTACCTCAG | 9444 F | 199 | | |
| | (SEQ ID NO: 47) | | | | |
| | CAGGTGATTGATACTCCTGATGCGA (SEQ ID NO: 48) | 9643 R | | | |
| ND3 | CATTTTGACTACCACAACTCAACGGCTAC (SEQ ID NO: 49) | 10120 F | 160 | | |
| | GGGTAAAAGGAGGGCAATTTCTAGATC (SEQ ID NO: 50) | 10280 R | | | |
| ND4L | GCTACTCTCATAACCCTCAACACCC (SEQ ID NO: 51) | 10599 F | 130 | | |
| | AGGCCATATGTGTTGGAGATTGAGA (SEQ ID NO: 52) | 10729 R | | | |
| ND4 | CCAACGCCACTTATCCAGTG (SEQ ID NO: 53) | 10999 F | 237 | | |
| | GGGAAGGGAGCCTACTAGGGTGT (SEQ ID NO: 54) | 11236 R | | | |
| ND5 | TTACCACCCTCGTTAACCCTAACAAA (SEQ ID NO: 55) | 12395 F | 165 | Real | |
| | TGGGTTGTTTGGGTTGTGGCT (SEQ ID NO: 56) | 12560 R | | Time/ | |
| | | | | PCR | |
| ND6 | ACGCCCATAATCATACAAAGCCC (SEQ ID NO: 57) | 14224 F | 149 | Real | |
| | GGATTGGTGCTGTGGGTGAAA (SEQ ID NO: 58) | 14373 R | | Time/ | |
| Cyt-b | CGCCTGCCTGATCCTCCAA (SEQ ID NO: 59) | 14860 F | 191 | PCR | |
| | AGGCCTCGCCCGATGTGTAG (SEQ ID NO: 60) | 15051 R | | | |
| Mito 1 | [aminoC6]ACATAGCACATTACAGTCAAATCCCTTCTCGTC | 16331 F | 3968 | Long | Dames, S., |
| | CCC (SEQ ID NO: 61) | | | PCR | Eilbeck, K. & |
| | [aminoC6]TGAGATTGTTTGGGCTACTGCTCGCAGTGC | 3729 R | | | Mao, R. A |
| | (SEQ ID NO: 62) | | | | high- |
| Mito 2 | [aminoC6]TACTCAATCCTCTGATCAGGGTGAGCATCAAAC | 3646 F | 5513 | | throughput |
| | TC (SEQ ID NO: 63) | | | | next- |
| | [aminoC6]GCTTGGATTAAGGCGACAGCGATTTCTAGGAT | 9458 R | | | generation |
| | AGT (SEQ ID NO: 64) | | | | sequencing |
| Mito 3 | [aminoC6]TCATTTTTATTGCCACAACTAACCTCCTCGGAC | 8753 F | 9289 | | assay for the |
| | TC (SEQ ID NO: 65) | | | | mitochondrial |
| | [aminoC6]CGTGATGTCTTATTTAAGGGGAACGTGTGGGCT | 16566 R | | | genome. |
| | AT (SEQ ID NO: 66) | | | | Methods in |
| | | | | | molecular |
| | | | | | biology 1264, |
| | | | | | 77-88 (2015) |

TABLE 3
Housekeeping Primers Human
Housekeeping Genes

| | | | Length | | | |
|---|---|---|---|---|---|---|
| Gene | Sequence | Position | (bps) | Assay | Target | Reference |
| ACTB | AGGATCTTCATGAGGTAGTCAGTCAG | 1358 F | 98 | Real Time | DNA | Clone |
| | (SEQ ID NO: 67) | | | | | Manager 9 |
| | CCACACTGTGCCCATCTACG | 1456 R | | | | (Sci-Ed |
| | (SEQ ID NO: 68) | | | | | Software) |
| GAPDH | CTCTGCTCCTCCTGTTCGAC | 5030 F | 126 | Real | DNA | SnapGene |
| | (SEQ ID NO: 69) | | | Time/PCR | | Viewer |
| | CGCCCGCGTCCGGCCTACACA | 5156 R | | | | Software |
| | (SEQ ID NO: 70) | | | | | |
| ACTB | ACCAACTGGGACGACATGGAG | 313 F | 379 | PCR | RNA | Clone |
| | (SEQ ID NO: 71) | | | | | Manager 9 |
| | GTGGTGGTGAAGCTGTAGCC | 692 R | | | | (Sci-Ed |
| | (SEQ ID NO: 72) | | | | | Software) |

TABLE 4
Housekeeping Primers Murine
Housekeeping Genes

| | | | Length | | | |
|---|---|---|---|---|---|---|
| Gene | Sequence | Position | (bps) | Assay | Target | Reference |
| GAPDH | AGCAGCCGCATCTTCTTGTGCAG | 38,619,737 | 226 | Real | DNA/ | SnapGene Viewer |
| | TG (SEQ ID NO: 73) | | | Time/PCR | RNA | Software |
| | GGCCTTGACTGTGCCGTTGAATTT | 38,619,962 | | | | |
| | (SEQ ID NO: 74) | | | | | |
| COX4 | ATTGGCAAGAGAGCCATTTCTAC | 160 | 100 | Real | DNA/ | Tan, A.S. et al. |
| | (SEQ ID NO: 75) | | | Time/PCR | RNA | Mitochondrial |
| | | | | | | genome acquisition |
| | | | | | | restores respiratory |
| | | | | | | function and |
| | | | | | | tumorigenic |
| | | | | | | potential of cancer |
| | | | | | | cells without |
| | | | | | | mitochondrial DNA. |
| | | | | | | Cell metabolism 21, |
| | | | | | | 81-94 (2015) |

### Example 2

### Mitochondrial Genome Identified in EVs from Patients with Therapy Resistant Breast Cancer

Given the growing importance of mitochondrial DNA (mtDNA) copy number to the evolution of cancers, particularly resistance to therapy, these features might be captured in circulating extracellular vesicles (EVs). Although, the mitochondrial ND1 gene was previously identified in the EVs of glioblastoma cell lines and astrocytes, the presence of mitochondrial DNA has not been examined in EVs from patients with breast cancer. In order to address this, EVs were isolated and characterized by electron microscopy and NanoSight analysis, which demonstrated cup-shaped membrane vesicles ˜140 nm in diameter from the plasma of patients with metastatic ER+breast cancer, who at the time of blood draw had HTR disease (FIG. 1, Panel a and Table 5. The findings revealed that 19/22 (˜86%) of these patients had EVs containing high levels of mtDNA, irrespective of tumor burden (FIG. 1a, mitochondrial ND1 gene copy number). Conversely, EVs isolated from the plasma of: 1) individuals who were never diagnosed with cancer (n=9); 2) patients who were newly diagnosed with early stage (I-II) ER+breast cancer (following tumor removal) and thus had no evidence of cancer (n=12) and 3) patients with de novo untreated stage IV cancer (n=6) had either undetectable or very low levels of ND1 mtDNA (FIG. 1, Panel a). These data suggest that the presence of the ND1 mtDNA in circulating EVs is selective for patients with HTR disease and not simply a reflection of metastatic disease burden. The presence of genomic DNA in EVs from cancer derived cell lines and patients with pancreatic cancer is known. However, in the cohort of patients with metastatic HTR disease, only 7/22 (32%) expressed the nuclear gene encoding GAPDH (FIG. 7, Panel a and Table 5). Not only was the ND1 mitochondrial gene expressed in circulating EVs from HTR patients, but also the complete mitochondrial genome, as determined by long-range PCR (3 sequential PCRs amplifying 3.9 kb, 5.5 kb and 7.8 kb amplicons encompassing the complete 16.6 kb circular mitochondrial genome) and by whole mtDNA genome PCR amplification of 46 amplicons (FIG. 1, Panel b and Panel c and FIG. 7, Panel b). Finally, increased ND1 level is found in association with disease progression from multiple mtDNA-ND1 level determinations in different stages of the disease of a patient derived EVs (FIG. 7, Panel c).

Table 5, below, shows the following: DNA copy number in Hormonal therapy resistant (HTR) breast cancer and different stages of the disease. 1. Subtype of Tumor: Invasive Lobular Carcinoma (ILC) or Invasive Ductal Carcinoma (IDC). Estrogen Receptor (ER) and Progesterone Receptor (PR) % Expression. 2. Disease Sites: Organs with metastatic disease. 3. Disease Volume: Low indicates <1% organ involvement; Hi indicates >10% organ involvement. 4. ND1 copy number/10 ng of DNA and STD. 5. GAPDH copy number/10 ng DNA and STD.

TABLE 5

| | Patient | | | Disease | ND1 C# | ND1 C# | GAPDH | GAPDH |
|---|---|---|---|---|---|---|---|---|
| | # | Subtype | Disease Sites | Volume | Mean | STD | C# Mean | C# STD |
| HTR | 21 | IDC ER70 PR0 | bone | high | ND | ND | 1.08 | 0.50 |
| Metastatic | 2 | ILC ER100 PR50 | pleura, skin | low | 3505.61 | 893.91 | 12.58 | 2.49 |
| | 24 | IDC ER90 PR5 | liver, bone, LN, brain, | high | 3006.89 | 188.26 | ND | ND |
| | 4 | ILC ER50 PR0 | LN, gastric | low | 2037.19 | 195.66 | 69.79 | 3.76 |
| | 5 | IDC ER60 PR0 | bone, LN | high | 1819.69 | 194.33 | 102.59 | 24.89 |
| | 51 | IDC ER80 PR0 | brain, bone, skin, LN | high | 1167.87 | 31.23 | 7.23 | 2.25 |
| | 10 | IDC ER80 PR10 | liver, bone | high | 990.82 | 57.42 | 117.19 | 14.26 |
| | 35 | IDC ER100 PR50 | pleura, bone | low | 837.71 | 188.90 | 1.65 | 0.52 |
| | 41 | ILC ER80 PR0 | bone, LN | high | 834.69 | 177.56 | 1.45 | 0.44 |
| | 20 | IDC ER95 PR60 | lung | high | 714.48 | 138.97 | 0.18 | 0.04 |
| | 9 | IDC ER30 PR0 | liver, bone, LN | high | 694.30 | 29.51 | 105.67 | 12.93 |
| | 8 | IDC ER80 PR10 | LN, bone, breast | high | 519.77 | 23.36 | 767.91 | 116.76 |
| | 7 | IDC ER20 PR0 | bone | low | 417.38 | 6.45 | 103.77 | 15.24 |
| | 36 | IDC ER50 PR0 | skin, liver | low | 390.35 | 15.74 | 7.74 | 1.68 |
| | 22 | IDC ER50 PR0 | bone, LN | low | 300.99 | 5.77 | ND | ND |
| | 15 | IDC ER90 PR80 | bone | low | 247.82 | 20.34 | 76.81 | 6.78 |
| | 18 | IDC ER15 PR0 | liver, pleura, skin, | high | 169.71 | 38.37 | 0.09 | 0.03 |
| | | | bone, LN | | | | | |
| | 19 | IDC ER80 PR50 | bone, lymph node LN | low | 85.30 | 17.75 | 0.26 | 0.04 |
| | 17 | IDC ER60 PR5 | pleura, bone, liver | high | 78.38 | 4.52 | 1.02 | 0.33 |
| | 46 | ILC ER30 PR0 | skin, peritoneum, LN | low | 74.91 | 16.92 | 2.96 | .15 |
| | 47 | IDC ER80 PR50 | lung, lymph node | low | 10.90 | 3.39 | 1.00 | 0.82 |
| | 39 | ILC ER60 PR10 | pleura, bone, gastric | high | 3.08 | 0.98 | ND | ND |
| | | | serosa | | | | | |
| De Novo | 30 | IDC ER10 PR0 | skin | low | ND | ND | ND | ND |
| Metastatic | 32 | IDC ER80 PR50 | bone, breast | high | 6.22 | 0.24 | 24.04 | 11.23 |
| | 38 | IDC ER100 PR10 | breast, bone, LN | high | 9.97 | 2.30 | ND | ND |
| | 40 | ILC ER70 PR0 | breast, liver, LN | high | 17.05 | 1.46 | 0.88 | 0.86 |
| | 37 | IDC ER95 PR0 | LN, gastric serosa, | low | 42.29 | 13.62 | 0.68 | 0.24 |
| | | | bone, breast | | | | | |
| | 34 | ILC ER80 PR80 | breast, peritoneum, | low | ND | ND | 0.06 | 0.06 |
| | | | bone | | | | | |
| Stage 1-2 | 6 | IDC ER70 PR20 | | | 7.08 | 4.23 | 1.10 | 0.78 |
| | 13 | IDC ER100 PR50 | | | 1.58 | 1.71 | 6.02 | 9.42 |
| | 16 | IDC ER20 PR0 | | | ND | ND | ND | ND |
| | 23 | IDC ER90 PR90 | | | 6.31 | 2.69 | ND | ND |
| | 25 | IDC ER100 PR90 | | | ND | ND | ND | ND |
| | 28 | IDC ER50 PR20 | | | ND | ND | ND | ND |
| | 29 | IDC ER80 PR0 | | | 42.08 | 7.51 | 8.09 | 2.23 |
| | 31 | IDC ER100 PR70 | | | ND | ND | ND | ND |
| | 41 | IDC ER90 PR80 | | | 2.83 | 0.47 | 415.96 | 169.74 |
| | 42 | IDC ER30 PR0 | | | 8.93 | 3.53 | ND | ND |
| | 49 | IDC ER60 PR10 | | | 2.77 | 2.35 | 0.35 | 0.19 |
| | 52 | IDC ER90 PR60 | | | ND | ND | 0.20 | 0.04 |

### Example 3

### Metabolic Features of Hormonal Therapy Sensitive -HTS- Versus Resistant-HTR-Disease

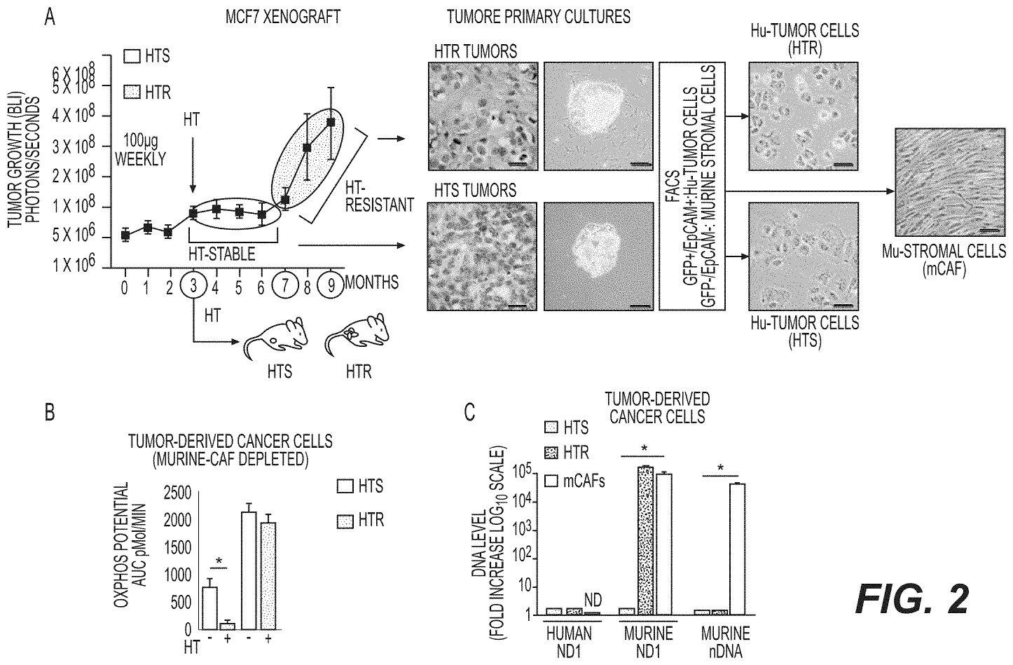

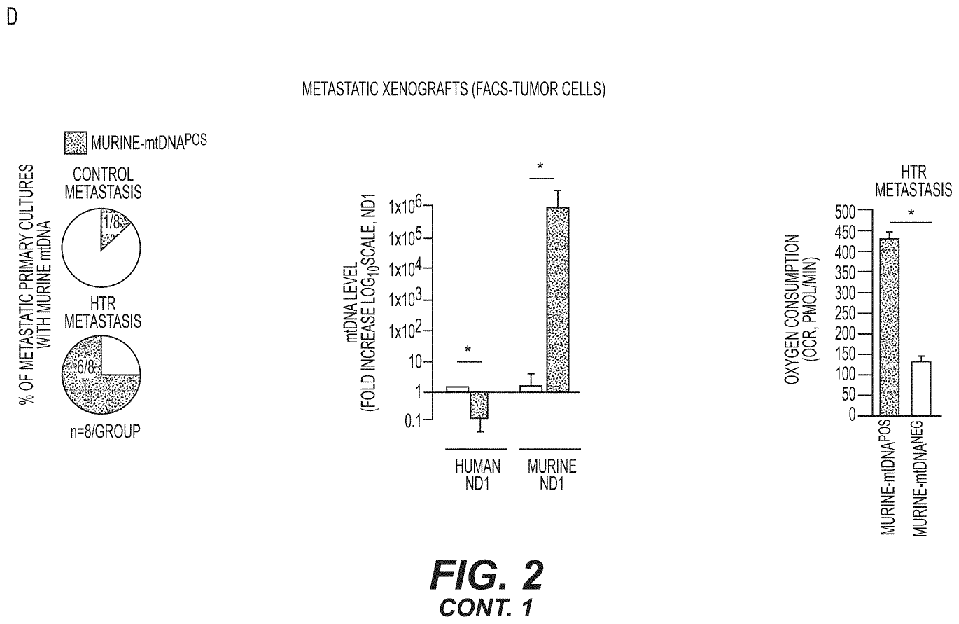

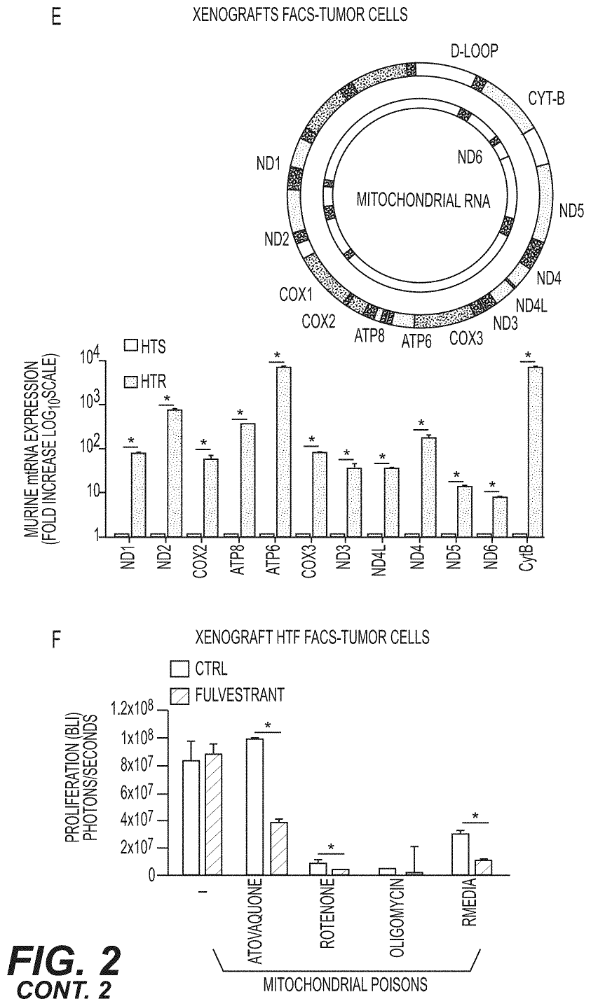

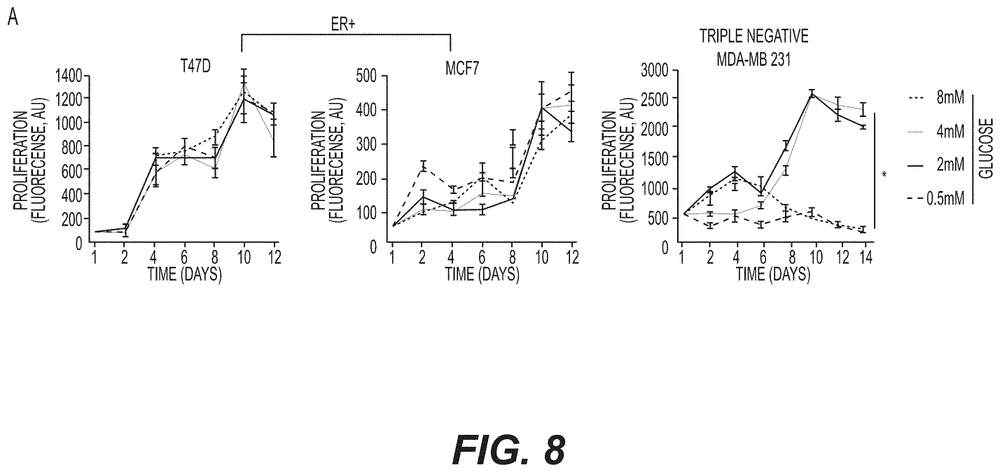

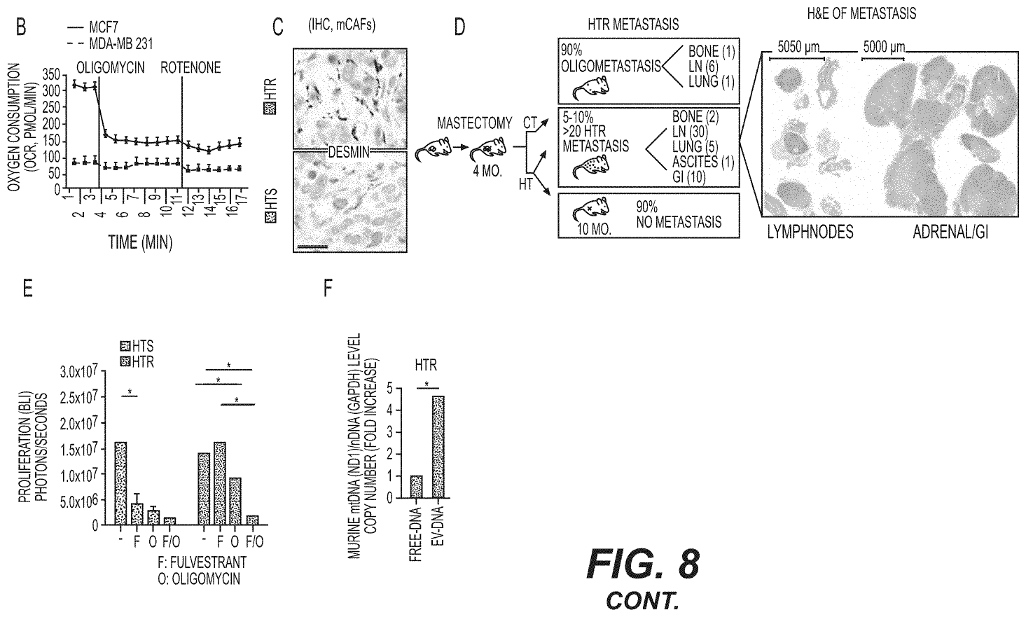

In order to study these phenomena in vivo, experimental models were developed that capture some of the essential features of the clinical scenario described above (the development of HTR disease). ER| (MCF7-Luciferase/GFP|) mammary fat pad xenografts were established in the absence of estradiol supplementation (Sansone, P. et al. Self-renewal of CD133(hi) cells by IL6/Notch3 signaling regulates endocrine resistance in metastatic breast cancer. Nature communications 7, 10442 (2016)). To identify the distinguishing characteristics of HTR disease, tumor-bearing mice were treated with HT (fulvestrant). Interestingly, a bimodal growth pattern was observed with no growth for 4 months (HTS) followed by exponential growth (HTR) (FIG. 2, Panel a). It has been demonstrated that luminal (ER+) breast cancers are metabolically dependent on oxidative phosphorylation (OXPHOS) rather than aerobic glycolysis (FIG. 8, Panel a- Panel b) (Sansone, P. et al. Self-renewal of CD133(hi) cells by IL6/Notch3 signaling regulates endocrine resistance in metastatic breast cancer. Nature communications 7, 10442 (2016); Martinez-Outschoorn, U. E., Peiris-Pages, M., Pestell, R. G., Sotgia, F. & Lisanti, M. P. Cancer metabolism: a therapeutic perspective. Nature reviews. Clinical oncology (2016)). The metabolic profile of HTR tumor cells might differ from HTS tumor cells. In order to test this, primary cultures from these two different tumor phases (HTS and HTR) were established and an enrichment of murine cancer associated fibroblasts (mCAFs) was noted in the HTR primary cultures as compared to HTS ones (FIG. 2, Panel a). Additionally, desmin immunohistochemical analysis of these HTR tissues confirmed the presence of CAFs, suggesting that the communication between cancer and stroma cells could instigate resistance to HT (FIG. 8, Panel c). By FACS analysis cancer cells (EpCAM+/GFP+) were separated from mCAFs (EpCAM−/GFP−) in these tumor specimens (FIG. 2, Panel a) and OXPHOS capacity was examined in these FACS sorted tumor-derived cells in the presence/absence of HT (fulvestrant). The OXPHOS potential of HTS cells was inhibited with HT, while HTR tumor-derived cells had a 3-fold greater OXPHOS capacity as compared to HTS cells, which was unaffected by HT (FIG. 2, Panel b).

### Example 4

### Host mtDNA Transfer in Xenograft Models of HTR Disease

Because depletion of mtDNA as well as mtDNA mutations abrogate OXPHOS function along with tumorigenic potential, HTR lesions could carry wild-type and higher mtDNA copy number, hence explaining their increased OXPHOS capacity. Detailed genetic characterization of mtDNA for the presence of mutations (see methods) was performed. mtDNA mutations were found in HTS cells, suggesting that reduced OXPHOS potential in HTS disease may be due to the presence of such mtDNA lesions (data not shown). In contrast, HTR cells harbored no such mutations and expressed murine mtDNA sequences (but not nuclear) to levels equal to that found in mCAFs, whereas there was no evidence of murine mtDNA in HTS tumor-derived cells (FIG. 2, Panel c). Additionally, HTS-derived xenograft cultures did not show outgrowth of mCAFs in vitro (FIG. 2, Panel a and data not shown).

In order to study the relevance of OXPHOS in the context of HTR metastatic disease, tumor-bearing mice (MCF7 model) underwent mastectomies followed by adjuvant HT (tamoxifen) or vehicle control and followed for ˜9 months. 100% of control mice had evidence of 1-2 metastases. Although 90% of the HT treated mice had no evidence of disease, 10% had wide-spread (>20) HTR metastases involving multiple organs (FIG. 8, Panel d). Cancer cells were isolated by FACS (for GFP) from HTR metastatic lesions and the presence of mtDNA was determined. The majority of HTR metastases as compared to naive metastatic disease (6/8 vs 1/8) expressed murine mtDNA (mu-mtDNA) (FIG. 2, Panel d). Interestingly, mu-mtDNA-expressing (mtDNAhi) HTR cells had a higher oxygen consumption rate compared to HTR cells lacking mu-mtDNA (FIG. 2, Panel d). Additionally, in agreement with a functional role of mu-mtDNA transfer, these mu-mtDNAhi HTR cells expressed the complete murine mtDNA-encoded transcriptome (FIG. 2, Panel e).

In order to determine the role of mitochondrial bioenergetics in HTR disease, mu-mtDNAhi HTR cells and HTS cells (no murine mtDNA) were treated with OXPHOS inhibitors (oligomycin, rotenone, atovaquone) with and without HT (fulvestrant). It was demonstrated that inhibitors of mitochondrial bioenergetics re-sensitized HTR cells to HT (FIG. 2, Panel f and FIG. 8, Panel e). On the contrary HTS cells displayed the same sensitivity to fulvestrant and mitochondrial inhibitors, suggesting no synergistic effect of OXPHOS inhibitors and HT on cell proliferation and/or cell death. Taken together, the data suggest that host (murine) derived mtDNA is acquired as cancer cells or metastases transition from an HT-sensitive state to one characterized by resistance to HT.

### Example 5

### CAF EVs Contain the Full Mitochondrial Genome

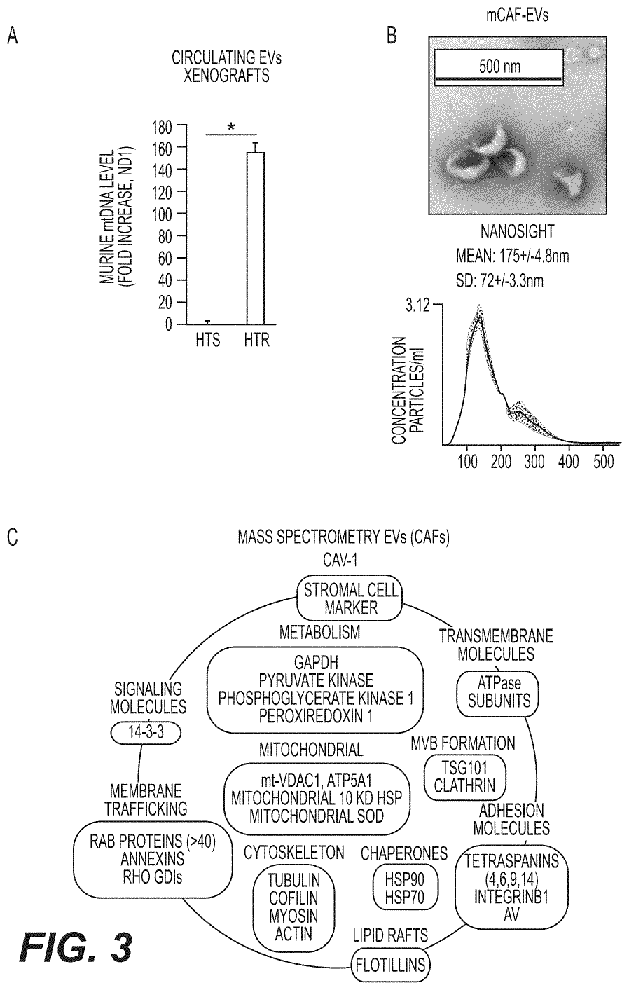

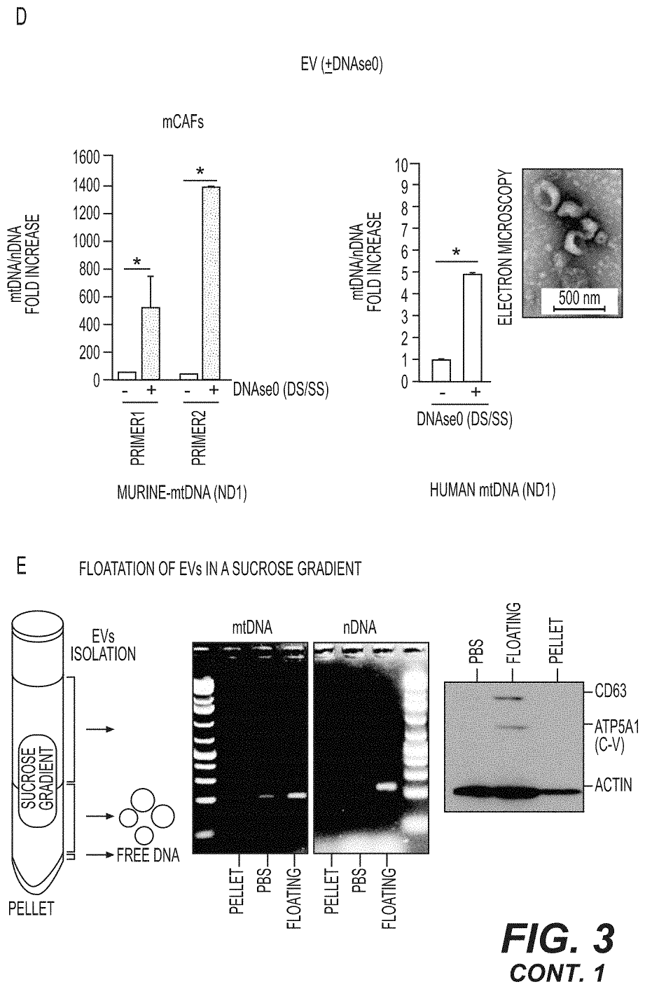

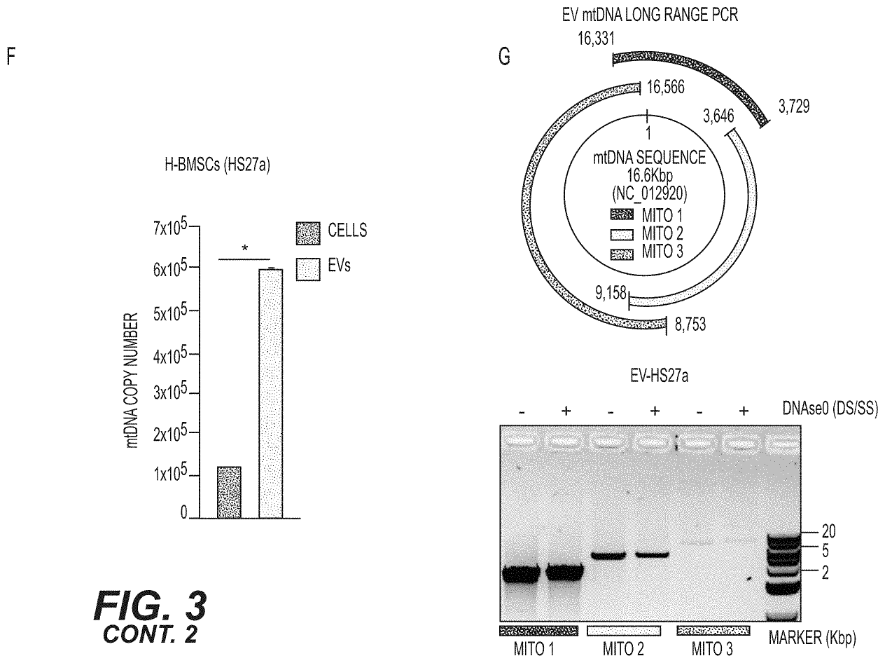

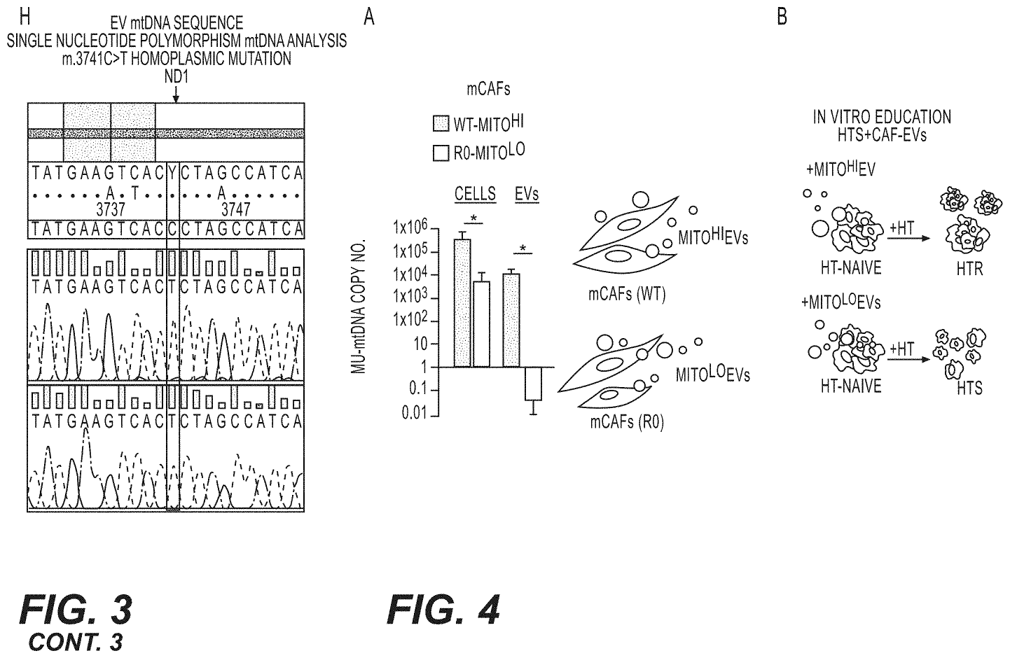

It is well accepted that CAFs and recruited bone marrow derived stromal cells play a critical role in cancer initiation, growth, invasion, metastasis and therapeutic resistance through the production of growth factors, cytokines, chemokines, catabolites, extracellular matrix proteins and EVs which modulate the behavior of cancers including their metabolism. Given that EVs have been shown to express and horizontally transfer genetic material (e.g. DNA, miRNA) to recipient cells, CAF-derived EVs could 1) harbor the mitochondrial genome and 2) transfer the mtDNA into the mitochondria of HT-treated tumor cells. It was demonstrated that mice harboring HTR xenografts had circulating EVs with a 150-fold higher murine mtDNA ND1 level as compared to HTS ones (FIG. 3, Panel a). Moreover, the ratio of mtDNA/genomic DNA was markedly elevated in these EVs (FIG. 8, Panel f).

EVs were isolated from the conditioned media of cultured murine CAFs (mCAFs) using a sucrose gradient approach. Electron microscopy and NanoSight analysis of these EVs demonstrated cup-shaped membrane vesicles ˜150 nm (FIG. 3, Panel b). Although these vesicles are larger than standard exosomes (-100nm), quantitative mass spectrometry of these CAF-derived EVs revealed together with the presence of numerous canonical proteins identified in exosomes (i.e. vesicles of endocytic origin) also some mitochondrial proteins such as ATP5A and VDAC1 (FIG. 3, Panel c)(Colombo, M., Raposo, G. & Thery, C. Biogenesis, secretion, and intercellular interactions of exosomes and other extracellular vesicles. Annual review of cell and developmental biology 30, 255-289 (2014)). By qPCR (using specific primers for mtDNA and nuclear DNA -nDNA-), it was determined that mtDNA was markedly enriched compared to nDNA in EVs as DNaseO treatment of EVs eliminated most of free DNA contamination but had no effect on mtDNA levels (FIG. 3, Panel d, see methods). Flotation of the 100,000 g pellets on sucrose gradients followed by EV isolation, DNA and western blot analyses further demonstrated that mtDNA is enriched in the CD63hi EV component (FIG. 3, Panel e). In agreement with the mass spectrometry data (FIG. 3, Panel c), it was confirmed the up-regulation of mitochondrial complex V subunit F0 (ATP5A1) in mtDNAhi/CD63hi EVs component.

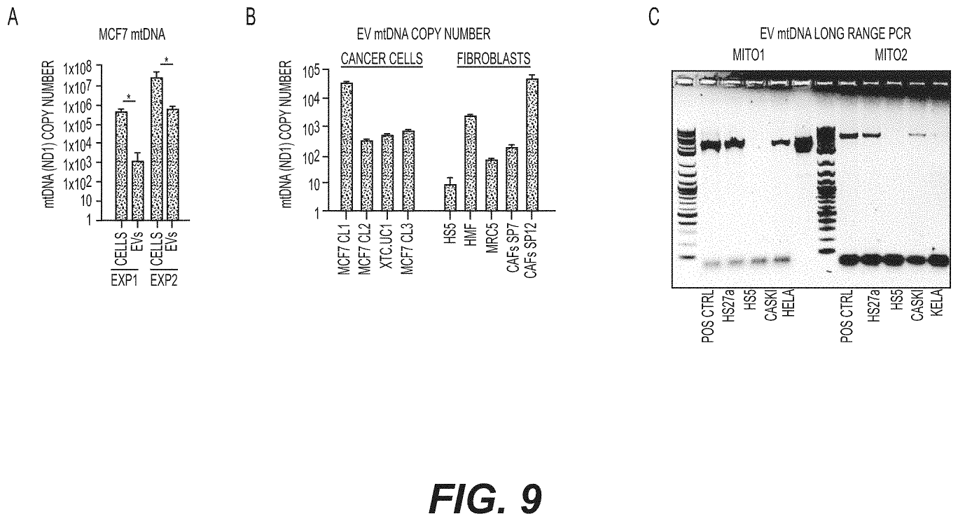

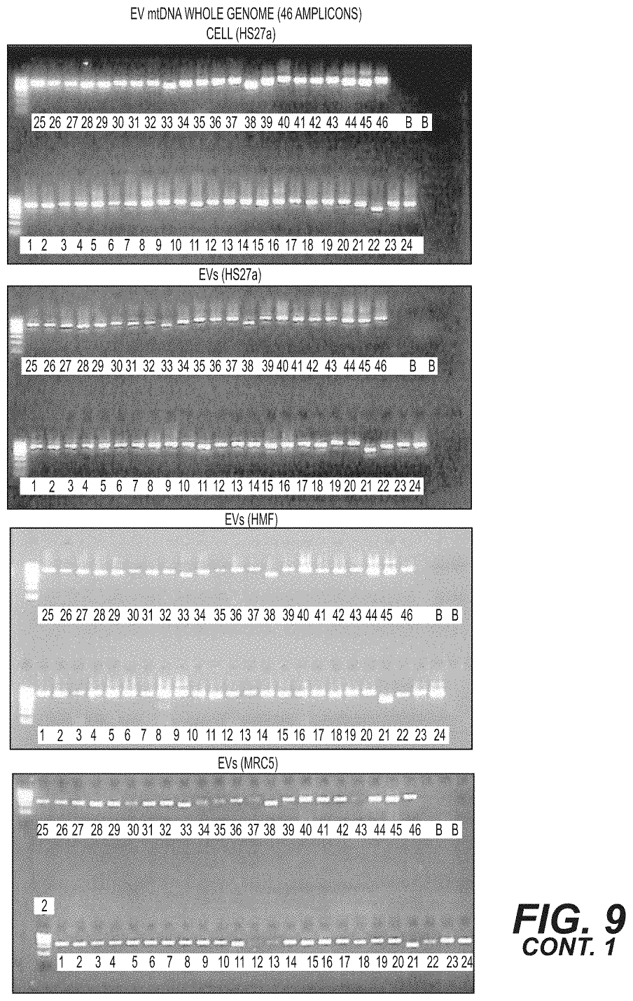

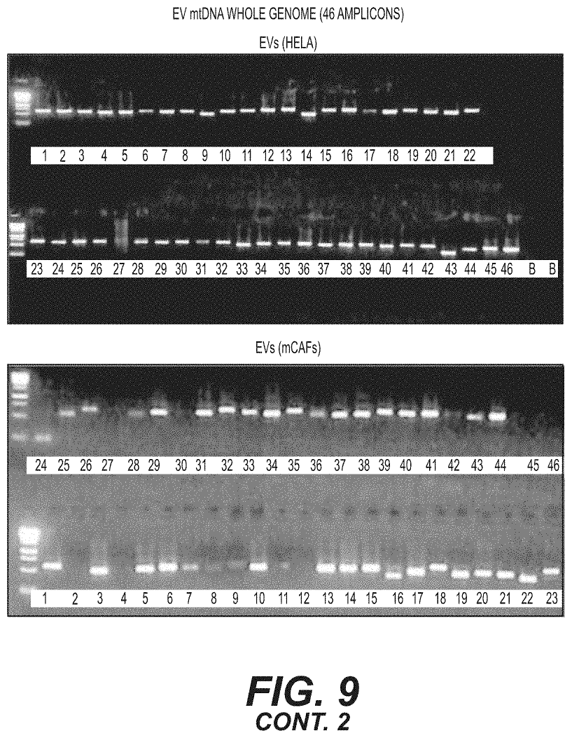

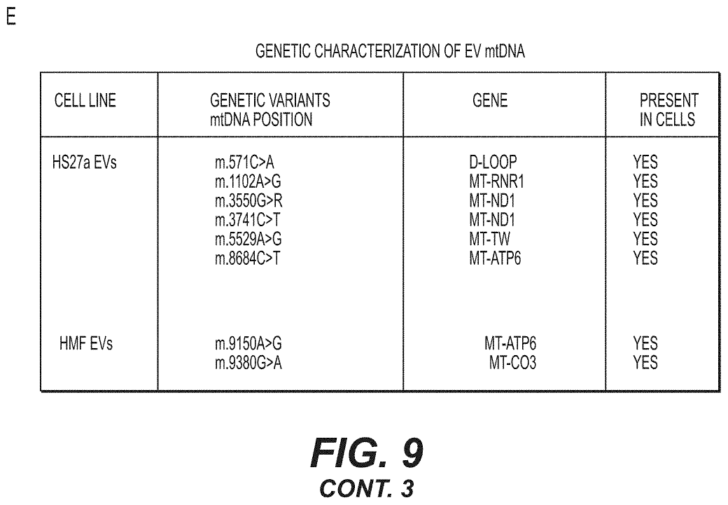

In addition to mCAFs, EVs were also examined from distinct human cell lines including cancer cell lines (Hela, Caski, MCF7), bone marrow derived stromal cell lines (HS27a, HS5), normal fibroblasts (HMF, MRC5) as well as patient derived CAFs primary cultures from bone metastases. The levels of mtDNA in EVs were proportional to cellular mtDNA expression (FIG. 9, Panel a). Using multiple approaches including absolute DNA copy number, long range PCR and 46-amplicon based PCR, the complete mitochondrial genome was identified packaged within EVs with the exception of those from HS5 cells (FIG. 9, Panel b,-Panel d). Amongst all the examined models, HS27a derived EVs expressed the highest levels of mtDNA relative to intracellular mtDNA (FIG. 3, Panel f, Panel g). Consistent with their cell of origin, mtDNA SNPs and mutations were also conserved between cells and their EVs, suggesting this process does not necessarily serve to simply clear damaged mtDNA genomes, e.g. after a mitophagic trigger (FIG. 3, Panel h and FIG. 9, Panel e). Taken together these data demonstrate the presence and packaging of the entire mitochondrial genome within many but not all cell-derived EVs.

### Example 6

### Functional Consequences of EV-mtDNA Transfer

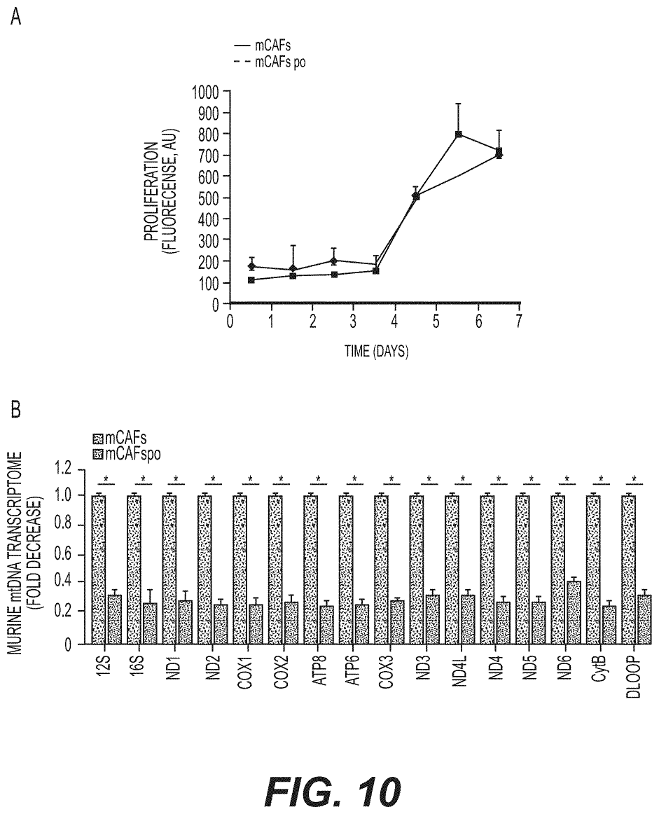

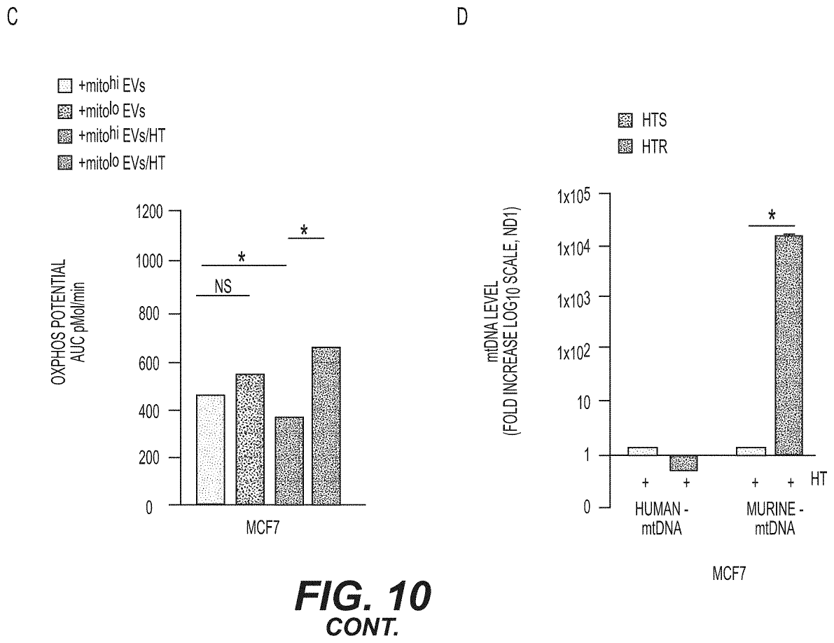

Given the observation that murine mitochondrial DNA was acquired in the transition from HTS to HTR tumors (FIG. 2) and the presence of the mitochondrial genome within CAF-EVs, EVs could transfer mtDNA to cancer cells promoting HTR disease. EVs were isolated from wild-type and mtDNA deficient CAFs cells using the ρ0 protocol (see methods). Notably, the depletion of mtDNA in CAFs did not affect their proliferation potential but resulted in a profound decrease in EV mtDNA copy number as well as mitochondrial transcripts (FIG. 4, Panel a and FIG. 10, Panel a, Panel b). To test the role of mtDNA transfer in mediating the transition from HTS to HTR disease, EVs were isolated from murine CAFs (wild type or ρ0) and administered them to HT-naïve cells weekly ×4 in the presence/absence of HT (FIG. 4, Panel b-schematic). The number of EVs administered (3×109) was determined experimentally (108-1011 EVs were tested) and represents the amount produced by ˜1×109 cells in 48 hours. No effect of EVs was observed in the absence of HT; while CAF-derived mitohi-EVs conferred HTR growth (FIG. 4, Panel b-c). Further characterization of the OXPHOS capacity of these cells demonstrated an increase in OXPHOS in those cells educated with mitohi-EVs whereas no effect was found in the HT treated cells educated with mitolo-EVs (FIG. 10, Panel c). In addition, the HTR mitohi EV educated cells contained murine (EV-derived) mtDNA (FIG. 10, Panel d). The transfer of mtDNA in vivo could promote HTR disease. HT-naive cells were injected into the MFP of mice followed by either weekly injections of CAF-derived mitohi-EVs or EVs lacking mtDNA (mitolo-EVs). After 2 months, no difference in tumor growth was observed in the two cohorts. However, 6 weeks following the administration of HT (fulvestrant), those mice educated with mitohi-EVs had developed tumors resistant to HT, while those educated with mitolo-EVs had evidence of HTS disease as their tumor burden decreased (FIG. 4, Panel d). Thus, it was demonstrated that CAF-EVs can transfer mtDNA conferring HTR disease both in vitro and in vivo.

### Example 7

### EV-mtDNA Transfer Mediates an Exit from Metabolic Dormancy

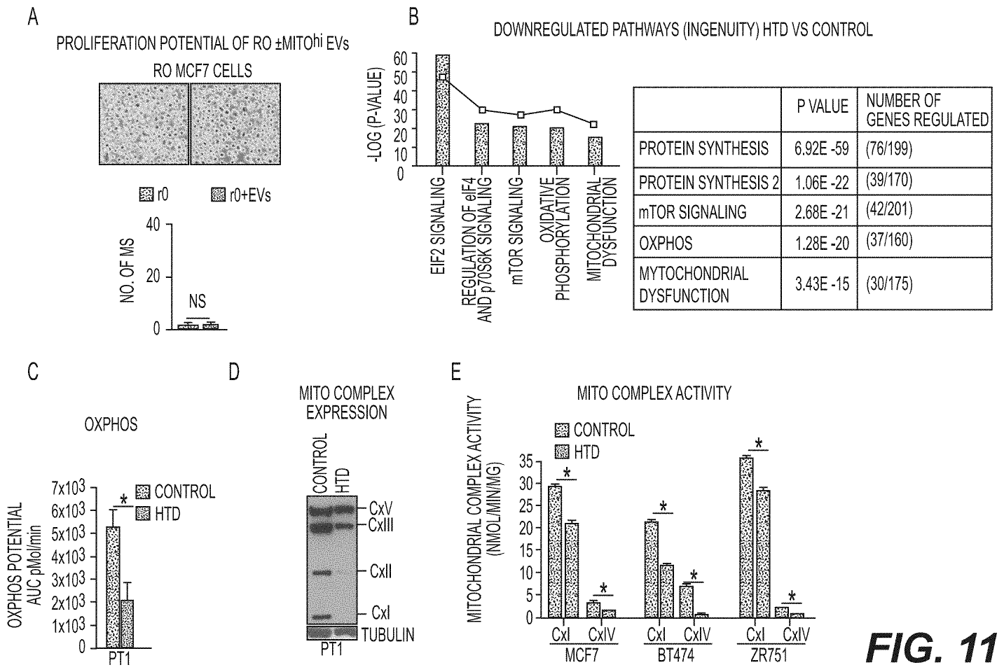

Because human mtDNA was reduced but still present following HT, murine mtDNA (mu-mtDNA) could alone rescue the loss of OXPHOS in cancer cells depleted for endogenous mtDNA. Luminal breast cancer cells depleted for mtDNA (ρo) were generated and educated with mtDNAhi CAF-EVs for 2 months. Not surprisingly, these ρo did not proliferate in vitro and in vivo nor did EV-mediated-mtDNA transfer rescue their proliferation potential (FIG. 11, Panel a and data not shown). This suggested that residual presence of endogenous mtDNA and nuclear encoded mitochondrial apparatus is necessary for the exogenous mtDNA-mediated proliferation potential of recipient cells.

EV mediated mtDNA transfer may also occur in dormant or metabolically quiescent tumor cells leading to an exit from dormancy. The recipient cells were first considered. It was known that HT (fulvestrant or tamoxifen) treatment of HT-naïve tumor cells led to the generation of cells that were metabolically OXPHOS low and expressed low levels of mtDNA.

Similarly, here ER+tumor-derived cells and cell lines were treated with vehicle or HT (fulvestrant) and isolated viable (DAP1-) cells by FACS (FIG. 5, Panel a). These HT-induced metabolically quiescent/dormant cells (HT-Dorm) 1) did not proliferate in culture but remained viable as single cells; 2) displayed decreased expression of genes related to protein synthesis and OXPHOS by microarray analysis (FIG. 11, Panel b). In several models of HT-induced tumor dormancy, it was demonstrated that mtDNA level (copy number) was reduced by 70-90% in the HT treated cells compared to control (FIG. 5, Panel b). Additionally, OXPHOS potential, mitochondrial complex protein expression and mitochondrial complex I and IV activity were also reduced in cells treated with HT (FIG. 11, Panel c- Panel e). Overall these data indicate that HT can induce an OXPHOS dormant phenotype characterized by the loss of mtDNA and mitochondrial complex activity.

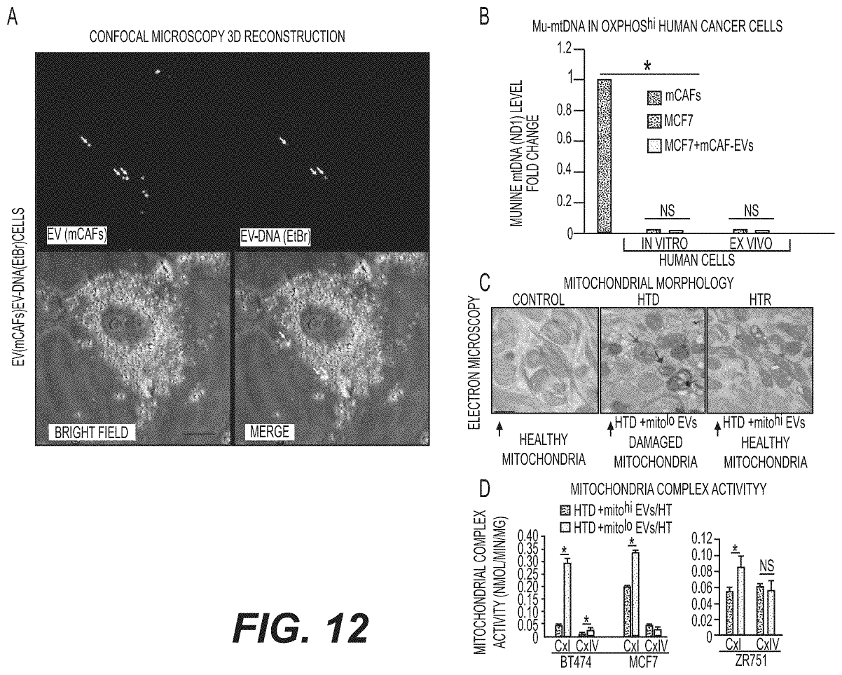

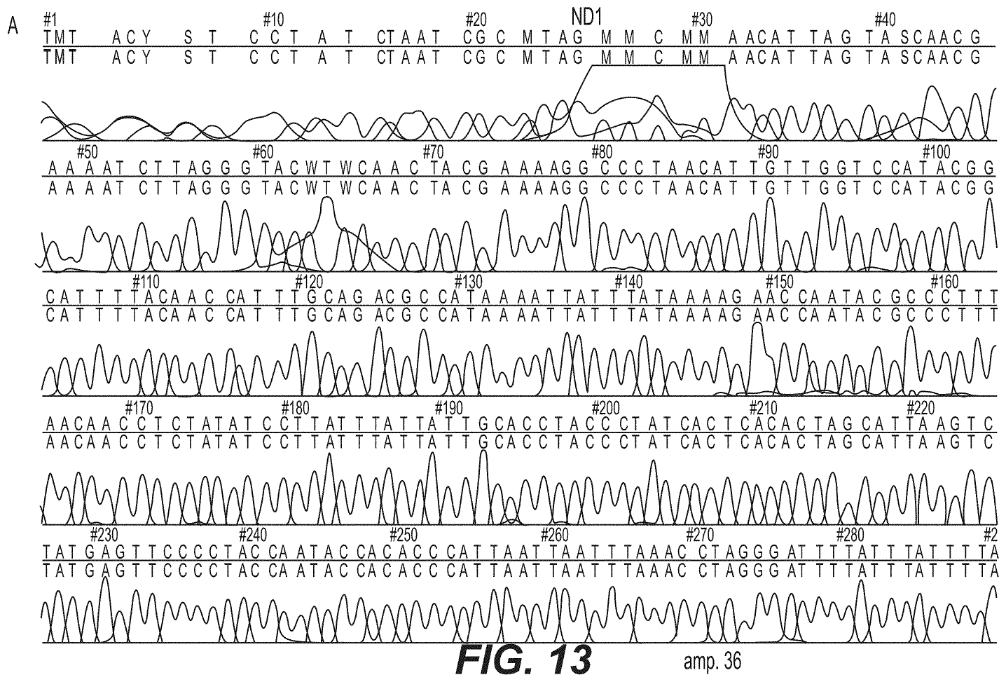

Given the observation that HTR disease harbors murine mtDNA (Fig.2), EVs could transfer their mtDNA into HT-dormant cells resulting in an exit from metabolic quiescence. EVs were isolated from murine CAFs (wild type or ρ0) and administered them to HT-dormant cells weekly ×4 in the presence of HT (FIG. 5, Panel c, schematic). After 6 weeks the proliferation of HT-Dorm cells as mammospheres (MS) with self-renewing capacity (HTR-EV cells) was observed (FIG. 5, Panel c). The functional consequences of the mtDNA transfer led to the activation of mitochondrial membrane potential (by TMRE staining) only observed in the HTR-EV cells (FIG. 5, Panel c). By confocal microscopy the co-localization of labeled EVs (PKH67-green) was observed with the mitochondria (mitotracker-red) 48 hours after their administration to HT-Dorm cells suggesting that EVs can fuse with recipient cell's mitochondria (FIG. 5, Panel d). Additionally, the majority of CAF-EVs harbor DNA which could be transfer to recipient cells (FIG. 12, Panel a). In contrast to HTD cells, OXPHOS proficient cancer cells (control cells never administered with HT) did not show the presence of mu-mtDNA following mtDNAhi EV administration in vitro and in vivo, suggesting that OXPHOS deficient cancer cell were more permissive to the replication and translation of exogenous mtDNA (FIG. 12, Panel b). In agreement with this observation, proliferating HTR-mitohi EV educated cells (MCF7 and BT474) demonstrated the presence of murine mtDNA and the expression of the complete murine mitochondrial transcriptome (FIG. 5, Panel e and Panel f). In multiple luminal breast cancer models of HT-Dorm to HTR (mitohi-EV educated) cells, increased mitochondrial complex I and IV activity was observed and restoration of normal appearing mitochondria (FIG. 12, Panel c and Panel d).

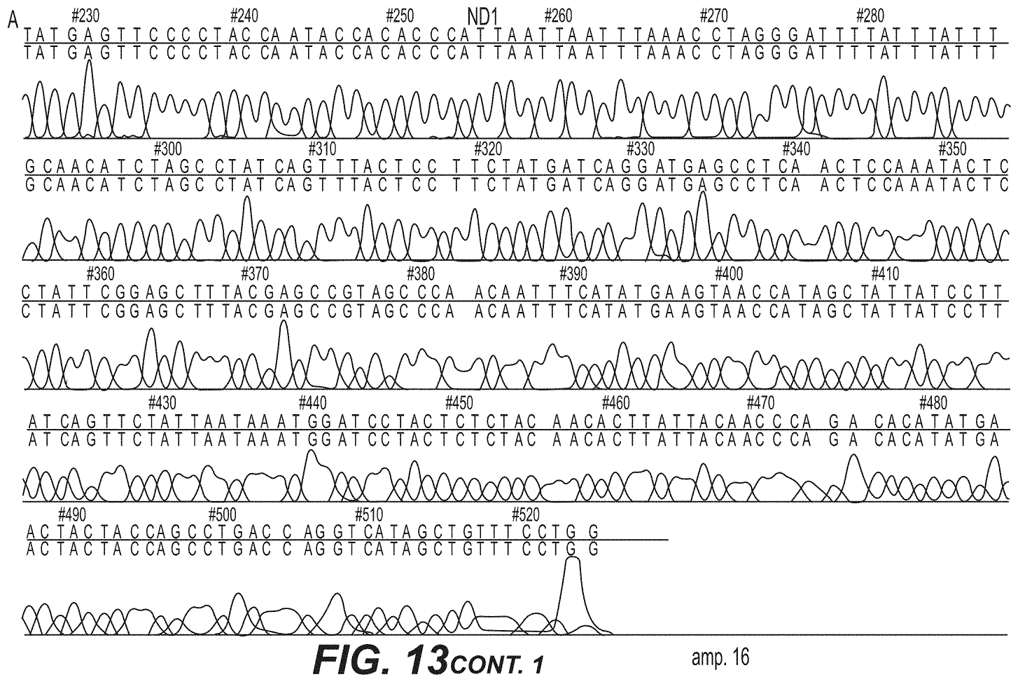

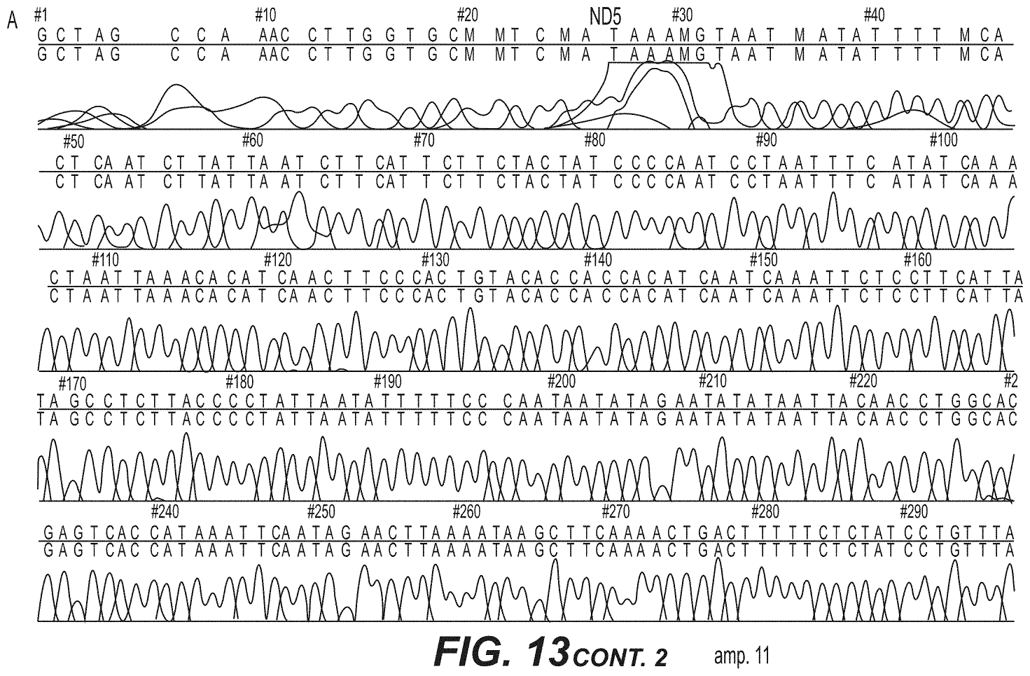

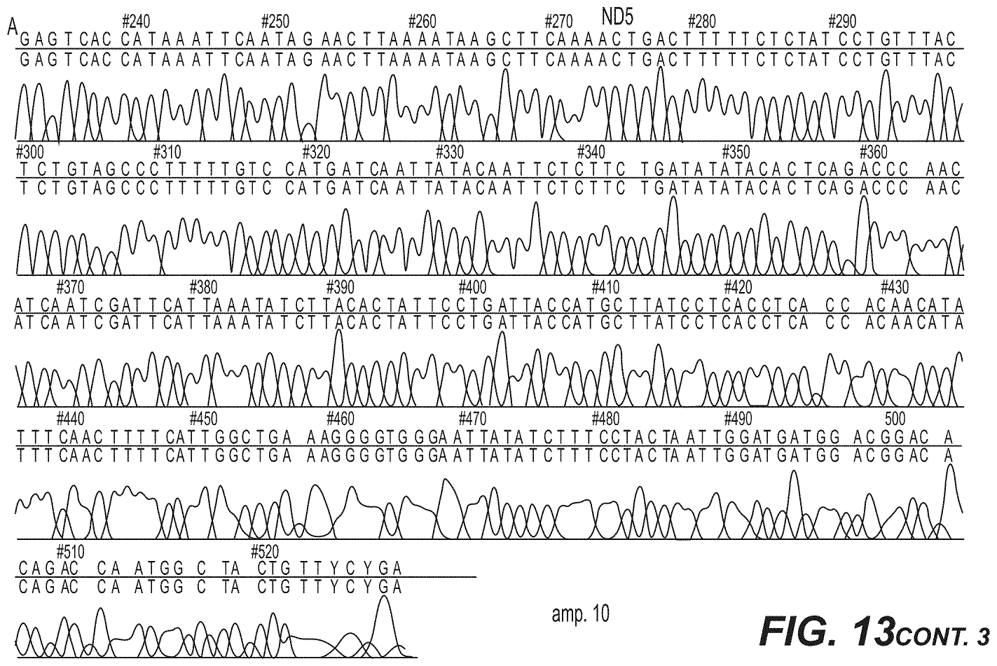

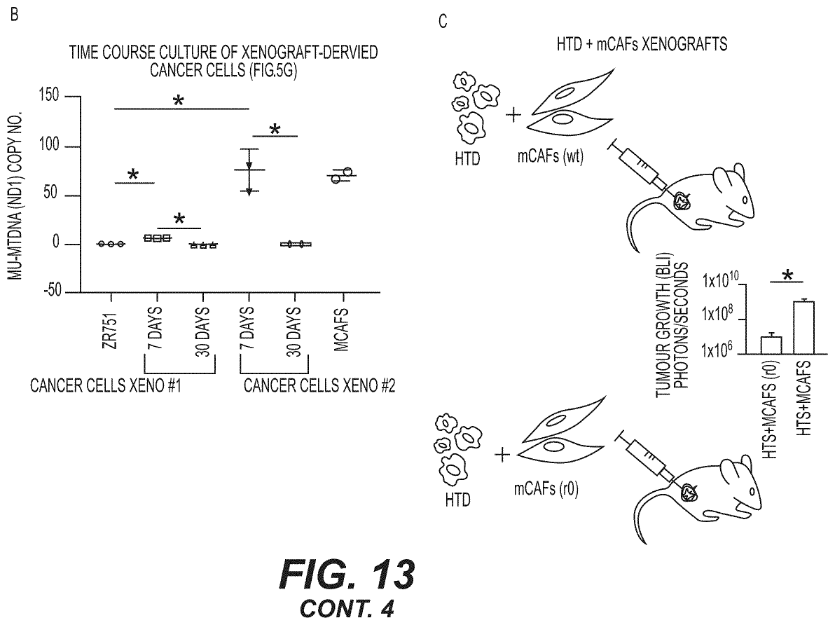

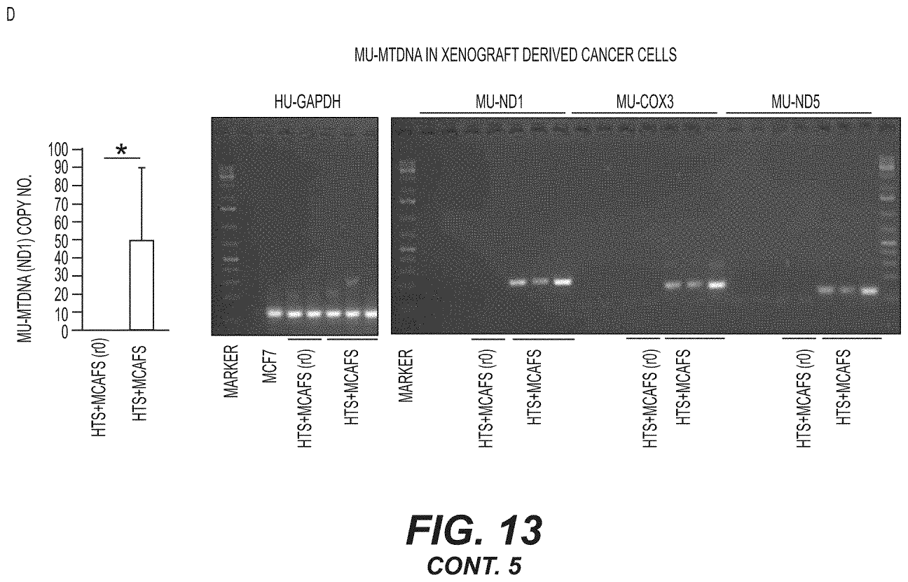

The transfer of mtDNA in vivo could promote exit from metabolic dormancy. HT-Dorm cells were injected into the MFP of mice followed by weekly injections of either CAF derived mitohi-EVs or EVs lacking mtDNA (mitolo-EVs). After 2 months 3/5 mice from the EV injected cohort had developed large tumors, while 2/5 of the control mice had very small tumors (FIG. 5, Panel g). Analysis of tumors from mitohi-EV educated mice demonstrated the presence of the whole murine mtDNA genome by PCR and sequencing analyses of the murine mtDNA only in FACS-purified tumor cells from mitohi-EVs educated xenografts (GFP+) (FIG. 5, Panel g and Panel h, FIG. 13, Panel a and data not shown). Higher murine mtDNA level could persist following ex vivo passages. Cancer cells isolated from mitohi-EVs xenografts were cultured from 7 to 120 days in culture and mtDNA level was determined every 7 days. It was found that mu-mtDNA was lost after 30 days of ex vivo cultures, suggesting that continuous EV education is needed for exogenous mtDNA transfer and activity (FIG. 13, Panel b).

Finally, it was tested whether the loss or decreased production of mtDNA (ρ0-CAFs) could interfere with the CAF-induced exit from dormancy of HT-Dorm cells (FIG. 12). Wild type mCAFs or ρ0mCAFs were co-injected with HT-Dorm cells in the MFP (MCF7-Luciferase/GFP+). After 6 months, 90% (9/10) of mice bearing mCAFs /HT-Dorm cells had tumors and metastases, whereas only in 17% (2/12) of mice harboring mtDNAloCAF/HT-Dorm cells generated tumors but developed no metastases (FIG. 13, Panel c). Analysis of cancer cells isolated from HT-Dorm cells, which had exited dormancy, expressed murine mtDNA as determined by ND1 mtDNA copy number and the presence of murine COX3 and ND5 mtDNA genes (FIG. 13, Panel d). Overall, these observations in multiple models of ER+breast cancer demonstrate that CAFs can package relatively large amounts of DNA—the full mitochondrial genome—into EVs which are released, taken up by cancer cells followed by the transcription of the donor mtDNA resulting in higher OXPHOS potential and exit from metabolic quiescence or “dormancy” and the development of HT-resistance.

### Example 8

### mtDNA Horizontal Transfer from EV to Cancer Cells Promote Therapy Resistant Breast Cancer

Since the discovery of the Warburg effect, many efforts have been made to study metabolic reprogramming in cancer cells from the gain of oncogenes, metastatic progression and in response to therapy. It was demonstrated that luminal breast cancer cells (≥70% of human breast cancer) require efficient mitochondrial respiration to maintain their tumorigenic potential. Both triple negative and Her2+ breast cancer cell lines are more dependent on anaerobic glycolysis, as depletion of mtDNA decreases their metastatic capacity, suggesting that functional respiration is required for metastatic dissemination. These observations are supported by the association between the occurrence of deleterious mtDNA mutations and a low tumorigenic phenotype. Conversely, genetic reconstitution of mitochondrial function (using cybrids) of mtDNA depleted cancer cells promoted reactive oxygen species (ROS) production leading to cell proliferation.

Cross-species exchange of mtDNA did not restore OXPHOS potential in xenocybrids. However, whether the transfer of mtDNA from a murine cell to a human cancer cell via EVs could rescue mitochondrial bioenergetics was never postulated before. Therefore, this model differs from xenocybrids for several reasons: 1) it does not use enucleated donor cells which function as “mitochondrial” donors containing full mitochondria, cytoplasm and organelles which fuse with recipient cells; 2) the recipient cells are not mtDNA depleted but have reduced mtDNA from hormonal therapy; 3) it performs experiments with EV which are vehicles capable of transferring genomic DNA from cell type to the next conferring profound phenotypes including transformation; 4) EVs fuse with resident mitochondria and are known to traffic to mitochondria via Rabs. The experimental model demonstrates the transfer of exogenous mtDNA in vivo and in vivo via EVs and provides its functional consequence in OXPHOS dependent breast cancers.

Mutations, deletions and changes in mtDNA copy number have been observed in cancers, particularly in response to therapy. However, the mechanisms and the clinical relevance of these phenomena remain unclear. These mutations may represent passenger events during therapy-driven cancer cell selection, alternatively they may be drivers of disease when accumulating towards homoplasmy playing a prominent role in chemo-resistance. While decreased mtDNA copy number reduces replication conferring resistance to antineoplastic drugs such as anthracyclines and taxanes, efficient mitochondria biogenesis would make cells more resistant to anti-mitochondrial agents as was demonstrated in melanomas treated with BRAF inhibitors.

The mechanisms by which the complete mitochondrial genome is selectively packaged within EVs are not known, although it was demonstrated that the cells most capable of producing mtDNAhiEVs are those that can readily reprogram their metabolism in response to oxidative stress resulting in reduced mitochondrial activity (OXPHOS) and increased mtDNA replication (this manuscript and data not shown). Most normal cells respond to oxidative stress, hypoxia, DNA damaging agents and/or nutrient deprivation by undergoing autophagy, apoptosis and senescence. Those cells that survive in these conditions or even continue to proliferate are those that are the optimal producers of functional mtDNA+EVs. It has been shown that mesenchymal stem cells in response to oxidative stress package depolarized mitochondria in very large microvesicles. Although the entire mitochondria were not found in the experiments (mass spectrometry and EM experiments), it is possible that mitochondria generate EVs which contain some mitochondria components including mtDNA. These mitochondria derived EVs are released and transfer their cargo in recipient cells. Indeed, some mitochondrial proteins were found by semi-quantitative mass spectrometry (FIG. 3, Panel c, e.g., ATP5A, VDAC1, mitochondrial phosphate carrier protein and mitochondrial peroxiredoxin-5) in addition to an abundance of proteins commonly found in vesicles of endocytic origin. Importantly, once EVs are taken-up by cells, it is not known how the mtDNA is incorporated within the mitochondria which would require traversing multiple membranes (EV and mitochondrial) possibly through the translocase of the outer/inner membrane complexes (TOM/TIM). The study provides an important platform to elucidate for further studies on how the full mtDNA genome is packaged, released, taken up and incorporated into recipient cells.

Recurrences of ER+ disease can be detected years to decades after the primary tumor diagnosis, suggesting that tumor cells have the capacity to remain dormant for prolonged periods of time. Determining how tumor cells are induced into a dormant state and the systemic cues that eventually cause them to exit dormancy, leading to hormone refractory metastatic disease are poorly understood in large part due to the paucity of clinical material and relevant animal tumor models. In the current study, such models were generated and identified the presence of mtDNA-EVs in the circulation of patients with HTR ER+ metastatic disease and in HTR tumor-bearing mice. It was demonstrated in pre-clinical models that: 1) although HT eradicates many cancer cells, some undergo a state of viable dormancy, characterized by the loss of mitochondrial DNA, 2) the development of HTR full-blown metastatic disease occurs through the restoration of mitochondrial function and 3) this may occur through the transfer of mtDNA via CAF-EVs.

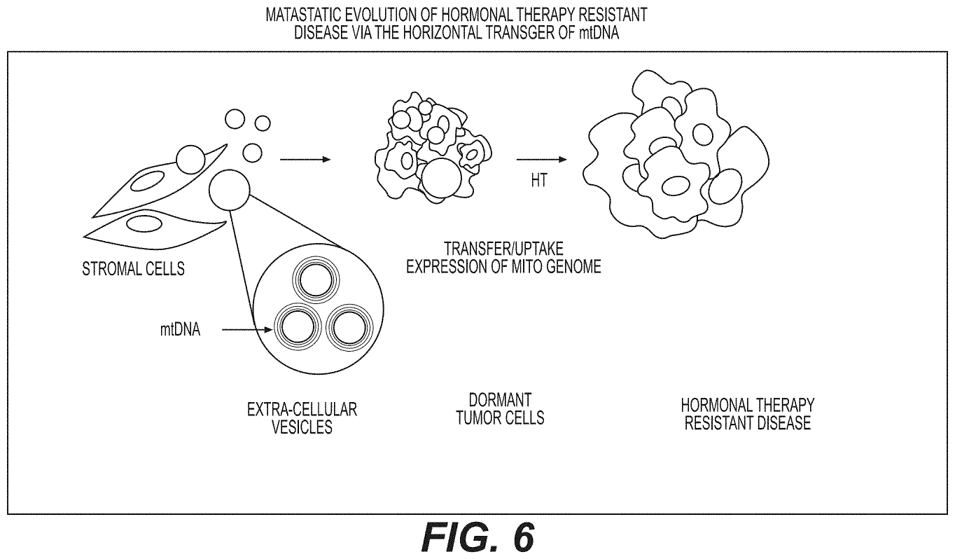

In summary, the data supports that in cancer: mtDNA+ extracellular vesicles are found and act as “infectious” mediators of therapy resistance in OXPHOS dependent human cancers leading to metastatic progression (FIG. 6).

The foregoing descriptions of specific embodiments of the present application have been presented for purposes of illustration and description. They are not intended to be exhaustive or to limit the application and method of use to the precise forms disclosed. Obviously many modifications and variations are possible in light of the above teaching. It is understood that various omissions or substitutions of equivalents are contemplated as circumstance may suggest or render expedient, but is intended to cover the application or implementation without departing from the spirit or scope of the claims of the present application.

1-15. (canceled)

16. A method for detecting a mtDNA related condition in a subject, comprising:
(a) isolating extracellular vesicles (EVs) from the plasma or other biological fluids, obtained from the subject;
(b) obtaining information of mtDNA in the isolated EVs; and
(c) determining a likelihood of presence or development of the mtDNA related condition based on the result of step (b).

17. The method of claim 16, wherein procedure (b) comprises detecting the levels of one or more mtDNA genes and/or 70%, 80%, 90% or 95% of full mtDNA genome in the isolated EVs, and wherein the mtDNA related condition is selected from the group consisting of cancer, metabolic disorders, heart failure, brain damage and neurologic syndromes.

18. The method of claim 17, further comprising:
(d) identifying specific genetic mtDNA variants; and
(e) determining the likelihood of presence or development of a mtDNA related condition based on results of procedures (b) and (d), wherein the mtDNA related condition is selected from the group consisting of cancer, metabolic disorders, heart failure, brain damage and neurologic syndromes.

19. The method of claim 16, wherein procedure (b) comprises identifying specific genetic mtDNA variants in the isolated EVs, and wherein the mtDNA related condition is selected from the group consisting of cancer, metabolic disorders, heart failure, brain damage and neurologic syndromes.

20. The method of claim 16, wherein procedure (b) comprises detecting the levels of one or more mtDNA genes and full mtDNA genome in the isolated EVs, and wherein the mtDNA related condition is a side effect of a radiation therapy or chemotherapy selected from the group consisting of neurological syndromes, heart failure, brain damage and lung/renal damage.

21. The method of claim 20, further comprising:
(d) identifying specific genetic mtDNA variants in the isolated EVs; and
(e) determining the likelihood of presence or development of the side effect of the radiation therapy or chemotherapy based on results of procedures (b) and (d).

22. The method of claim 16, wherein (b) comprises identifying specific genetic mtDNA variants in the isolated EVs, and wherein the mtDNA related condition is a side effect of a radiation or chemotherapy selected from the group consisting of neurological syndromes, heart failure, brain damage and lung/renal damage.

23. The method of claim 16, wherein procedure (b) comprises detecting the levels of one or more mtDNA genes and full mtDNA genome in the isolated EVs, and wherein the mtDNA related condition is resistance to a hormonal therapy in a breast cancer patient.

24. The method of claim 23, further comprising
(d) identifying specific genetic mtDNA variants,
wherein the likelihood of presence or development of resistance to a hormonal therapy in a breast cancer patient is determined based on results of procedures (b) and (d).

25. The method of claim 16, wherein procedure (b) comprises identifying specific genetic mtDNA variants in the isolated EVs, and wherein the mtDNA related condition is resistance to a hormonal therapy in a breast cancer patient.

26. The method of claim 16, wherein procedure (b) comprises detecting the levels of one or more mtDNA genes and/or 70%, 80%, 90% or 95% of full mtDNA genome in the isolated EVs, and wherein the mtDNA related condition is breast cancer.

27. The method of claim 16, wherein procedure (b) comprises identifying specific genetic mtDNA variants in the isolated EVs, and wherein the mtDNA related condition is breast cancer.

28. A method for treating, or reducing the likelihood of, a mtDNA related condition in a subject, comprising:
(a) isolating extracellular vesicles (EVs) from the plasma or other biological fluids, obtained from the subject;
(b) obtaining information of mtDNA in the isolated EVs; and
(c) determining whether the subject has an enhanced likelihood of presence or development of the mtDNA related condition based on the result of step (b); and
(d) if it is determined in (c) that the subject has an enhanced likelihood of presence or development of the mtDNA related condition, administering to the subject an effective amount of an agent that inhibits horizontal transfer of mtDNA.

29. The method of claim 28, wherein the mtDNA related condition is resistance to a hormonal therapy, and wherein the subject suffers from breast cancer.

30. The method of claim 29, wherein the subject suffers from metastatic breast cancer.

31. A diagnostic kit for diagnosing a mtDNA-related condition in a subject, the kit comprising:
(a) a filter for isolating EVs from a biological sample;
(b) reagents for isolating DNA from isolated EVs; and
(c) reagents for detecting one or more mtDNA markers in isolated DNA.

32. The kit of claim 31, wherein the reagents for detecting one or more mtDNA markers in isolated DNA comprise one or more DNA primers listed in Tables 1-4.

33. The kit of claim 31, wherein the reagents for detecting one or more mtDNA markers in isolated DNA comprise one or more DNA primers listed in Tables 2 and 3.


SEQUENCE LISTING (75 sequences)

SEQ ID NO: 1 - DNA, 20 bp, Artificial Sequence
  Synthetic oligonucleotide
aggtttggtcctggccttat

SEQ ID NO: 2 - DNA, 20 bp, Artificial Sequence
  Synthetic oligonucleotide
gtggctaggcaaggtgtctt

SEQ ID NO: 3 - DNA, 19 bp, Artificial Sequence
  Synthetic oligonucleotide
ctagaaaccccgaaccaaa

SEQ ID NO: 4 - DNA, 20 bp, Artificial Sequence
  Synthetic oligonucleotide
ccagctatcaccaagctcgt

SEQ ID NO: 5 - DNA, 20 bp, Artificial Sequence
  Synthetic oligonucleotide
cagccggcccattcgcgtta

SEQ ID NO: 6 - DNA, 22 bp, Artificial Sequence
  Synthetic oligonucleotide
agcggaagcgtggataggatgc

SEQ ID NO: 7 - DNA, 24 bp, Artificial Sequence
  Synthetic oligonucleotide
tcctcctggccatcgtactcaact

SEQ ID NO: 8 - DNA, 21 bp, Artificial Sequence
  Synthetic oligonucleotide
agaagtggaatggggcgaggc

SEQ ID NO: 9 - DNA, 20 bp, Artificial Sequence
  Synthetic oligonucleotide
ccagtgctagccgcaggcat

SEQ ID NO: 10 - DNA, 25 bp, Artificial Sequence
  Synthetic oligonucleotide
tctgggtgcccaaagaatcagaaca

SEQ ID NO: 11 - DNA, 25 bp, Artificial Sequence
  Synthetic oligonucleotide
agttgataaccgagtcgttctgcca

SEQ ID NO: 12 - DNA, 21 bp, Artificial Sequence
  Synthetic oligonucleotide
tcggcctgggatggcatcagt

SEQ ID NO: 13 - DNA, 24 bp, Artificial Sequence
  Synthetic oligonucleotide
atgccacaactagatacatcaaca

SEQ ID NO: 14 - DNA, 20 bp, Artificial Sequence
  Synthetic oligonucleotide
ggggtaatgaatgaggcaaa

SEQ ID NO: 15 - DNA, 24 bp, Artificial Sequence
  Synthetic oligonucleotide
gctctcactcgcccacttccttcc

SEQ ID NO: 16 - DNA, 24 bp, Artificial Sequence
  Synthetic oligonucleotide
gccggactgctaatgccattggtt

SEQ ID NO: 17 - DNA, 23 bp, Artificial Sequence
  Synthetic oligonucleotide
acctaccaaggccaccacactcc

SEQ ID NO: 18 - DNA, 25 bp, Artificial Sequence
  Synthetic oligonucleotide
gcagcctcctagatcatgtgttggt

SEQ ID NO: 19 - DNA, 21 bp, Artificial Sequence
  Synthetic oligonucleotide
accctacaagctctgcacgcc

SEQ ID NO: 20 - DNA, 26 bp, Artificial Sequence
  Synthetic oligonucleotide
gctcatggtagtgaagtagaagggca

SEQ ID NO: 21 - DNA, 26 bp, Artificial Sequence
  Synthetic oligonucleotide
tcgctcccacctaatatccacattgc

SEQ ID NO: 22 - DNA, 23 bp, Artificial Sequence
  Synthetic oligonucleotide
gcaggctgcgaaaaccaagatgg

SEQ ID NO: 23 - DNA, 24 bp, Artificial Sequence
  Synthetic oligonucleotide
tcgcctactcctcagttagccaca

SEQ ID NO: 24 - DNA, 23 bp, Artificial Sequence
  Synthetic oligonucleotide
tgatgatgtgaggccatgtgcga

SEQ ID NO: 25 - DNA, 20 bp, Artificial Sequence
  Synthetic oligonucleotide
tcggaagcctcgccctcaca

SEQ ID NO: 26 - DNA, 22 bp, Artificial Sequence
  Synthetic oligonucleotide
agtagggctcaggcgttggtgt

SEQ ID NO: 27 - DNA, 26 bp, Artificial Sequence
  Synthetic oligonucleotide
aatacccgcaaacaaagatcacccag

SEQ ID NO: 28 - DNA, 26 bp, Artificial Sequence
  Synthetic oligonucleotide
tgttggggttatgttagagggaggga

SEQ ID NO: 29 - DNA, 19 bp, Artificial Sequence
  Synthetic oligonucleotide
acagcaaacggagcctcaa

SEQ ID NO: 30 - DNA, 23 bp, Artificial Sequence
  Synthetic oligonucleotide
tgctgtggctatgactgcgaaca

SEQ ID NO: 31 - DNA, 25 bp, Artificial Sequence
  Synthetic oligonucleotide
tggccacagcacttaaacacatctc

SEQ ID NO: 32 - DNA, 32 bp, Artificial Sequence
  Synthetic oligonucleotide
gggttgtattgatgagattagtagtatgggag

SEQ ID NO: 33 - DNA, 21 bp, Artificial Sequence
  Synthetic oligonucleotide
cccgttccagtgagttcaccc

SEQ ID NO: 34 - DNA, 19 bp, Artificial Sequence
  Synthetic oligonucleotide
tgtgggggtgccctttgtc

SEQ ID NO: 35 - DNA, 23 bp, Artificial Sequence
  Synthetic oligonucleotide
aactttgcaaggagagccaaagc

SEQ ID NO: 36 - DNA, 23 bp, Artificial Sequence
  Synthetic oligonucleotide
gggatttagagggttctgtgggc

SEQ ID NO: 37 - DNA, 24 bp, Artificial Sequence
  Synthetic oligonucleotide
acgccataaaactcttcaccaaag

SEQ ID NO: 38 - DNA, 26 bp, Artificial Sequence
  Synthetic oligonucleotide
tagtagaagagcgatggtgagagcta

SEQ ID NO: 39 - DNA, 24 bp, Artificial Sequence
  Synthetic oligonucleotide
cttctgagtcccagaggttaccca

SEQ ID NO: 40 - DNA, 26 bp, Artificial Sequence
  Synthetic oligonucleotide
ccgtcaactccacctaatttggtttg

SEQ ID NO: 41 - DNA, 23 bp, Artificial Sequence
  Synthetic oligonucleotide
tgccataacccaataccaaacgc

SEQ ID NO: 42 - DNA, 32 bp, Artificial Sequence
  Synthetic oligonucleotide
ctgttagtagtatagtgatgccagcagctagg

SEQ ID NO: 43 - DNA, 26 bp, Artificial Sequence
  Synthetic oligonucleotide
ctacggtcaatgctctgaaatctgtg

SEQ ID NO: 44 - DNA, 28 bp, Artificial Sequence
  Synthetic oligonucleotide
gctaagttagctttacagtgggctctag

SEQ ID NO: 45 - DNA, 27 bp, Artificial Sequence
  Synthetic oligonucleotide
gaaaatctgttcgcttcattcattgcc

SEQ ID NO: 46 - DNA, 25 bp, Artificial Sequence
  Synthetic oligonucleotide
gctgattagtggtgggttgttactg

SEQ ID NO: 47 - DNA, 31 bp, Artificial Sequence
  Synthetic oligonucleotide
cgatacgggataatcctatttattacctcag

SEQ ID NO: 48 - DNA, 25 bp, Artificial Sequence
  Synthetic oligonucleotide
caggtgattgatactcctgatgcga

SEQ ID NO: 49 - DNA, 29 bp, Artificial Sequence
  Synthetic oligonucleotide
cattttgactaccacaactcaacggctac

SEQ ID NO: 50 - DNA, 27 bp, Artificial Sequence
  Synthetic oligonucleotide
gggtaaaaggagggcaatttctagatc

SEQ ID NO: 51 - DNA, 25 bp, Artificial Sequence
  Synthetic oligonucleotide
gctactctcataaccctcaacaccc

SEQ ID NO: 52 - DNA, 25 bp, Artificial Sequence
  Synthetic oligonucleotide
aggccatatgtgttggagattgaga

SEQ ID NO: 53 - DNA, 20 bp, Artificial Sequence
  Synthetic oligonucleotide
ccaacgccacttatccagtg

SEQ ID NO: 54 - DNA, 23 bp, Artificial Sequence
  Synthetic oligonucleotide
gggaagggagcctactagggtgt

SEQ ID NO: 55 - DNA, 26 bp, Artificial Sequence
  Synthetic oligonucleotide
ttaccaccctcgttaaccctaacaaa

SEQ ID NO: 56 - DNA, 21 bp, Artificial Sequence
  Synthetic oligonucleotide
tgggttgtttgggttgtggct

SEQ ID NO: 57 - DNA, 23 bp, Artificial Sequence
  Synthetic oligonucleotide
acgcccataatcatacaaagccc

SEQ ID NO: 58 - DNA, 21 bp, Artificial Sequence
  Synthetic oligonucleotide
ggattggtgctgtgggtgaaa

SEQ ID NO: 59 - DNA, 19 bp, Artificial Sequence
  Synthetic oligonucleotide
cgcctgcctgatcctccaa

SEQ ID NO: 60 - DNA, 20 bp, Artificial Sequence
  Synthetic oligonucleotide
aggcctcgcccgatgtgtag

SEQ ID NO: 61 - DNA, 36 bp, Artificial Sequence
  Synthetic oligonucleotide
acatagcacattacagtcaaatcccttctcgtcccc

SEQ ID NO: 62 - DNA, 30 bp, Artificial Sequence
  Synthetic oligonucleotide
tgagattgtttgggctactgctcgcagtgc

SEQ ID NO: 63 - DNA, 35 bp, Artificial Sequence
  Synthetic oligonucleotide
tactcaatcctctgatcagggtgagcatcaaactc

SEQ ID NO: 64 - DNA, 35 bp, Artificial Sequence
  Synthetic oligonucleotide
gcttggattaaggcgacagcgatttctaggatagt

SEQ ID NO: 65 - DNA, 35 bp, Artificial Sequence
  Synthetic oligonucleotide
tcatttttattgccacaactaacctcctcggactc

SEQ ID NO: 66 - DNA, 35 bp, Artificial Sequence
  Synthetic oligonucleotide
cgtgatgtcttatttaaggggaacgtgtgggctat

SEQ ID NO: 67 - DNA, 26 bp, Artificial Sequence
  Synthetic oligonucleotide
aggatcttcatgaggtagtcagtcag

SEQ ID NO: 68 - DNA, 20 bp, Artificial Sequence
  Synthetic oligonucleotide
ccacactgtgcccatctacg

SEQ ID NO: 69 - DNA, 20 bp, Artificial Sequence
  Synthetic oligonucleotide
ctctgctcctcctgttcgac

SEQ ID NO: 70 - DNA, 21 bp, Artificial Sequence
  Synthetic oligonucleotide
cgcccgcgtccggcctacaca

SEQ ID NO: 71 - DNA, 21 bp, Artificial Sequence
  Synthetic oligonucleotide
accaactgggacgacatggag

SEQ ID NO: 72 - DNA, 20 bp, Artificial Sequence
  Synthetic oligonucleotide
gtggtggtgaagctgtagcc

SEQ ID NO: 73 - DNA, 25 bp, Artificial Sequence
  Synthetic oligonucleotide
agcagccgcatcttcttgtgcagtg

SEQ ID NO: 74 - DNA, 24 bp, Artificial Sequence
  Synthetic oligonucleotide
ggccttgactgtgccgttgaattt

SEQ ID NO: 75 - DNA, 23 bp, Artificial Sequence
  Synthetic oligonucleotide
attggcaagagagccatttctac